(19) United States
(12) Patent Application Publication
(10) Pub. No.: US 2023/0270072 A1
(43) Pub. Date: Aug. 31, 2023

(54) ALSTROEMERIA PLANTS WITH DOUBLE-TYPE FLOWERS WITH STABLE PRODUCTION

(71) Applicant: Van Zanten Breeding B.V., Rijsenhout (NL)

(72) Inventors: Willem Haytink, Rijsenhout (NL); Aart Van Voorst, Rijsenhout (NL)

(21) Appl. No.: 18/067,477

(22) Filed: Dec. 16, 2022

Related U.S. Application Data
(60) Provisional application No. 63/290,311, filed on Dec. 16, 2021.
(60) Provisional application No. 63/379,914, filed on Oct. 18, 2022.

(51) Int. Cl. A01H 6/56; A01H 5/02; A01H 1/04

(52) U.S. Cl. CPC A01H 6/564; A01H 5/02; A01H 1/045

(57) ABSTRACT

The present invention relates to plants from the genus Alstroemeria L. with a new flower type. Specifically, the invention thus relates to an Alstroemeria L. plant, which may comprise a single flower per stem, which flower may comprise more than 6 tepals. This new trait is called herein “double-type flower”.

### RELATED APPLICATIONS AND INCORPORATION BY REFERENCE

This application claims priority to US provisional patent application Ser. Nos. 63/290,311 filed Dec. 16, 2021 and 63/379,914 filed Oct. 18, 2022.

The foregoing applications, and all documents cited therein or during their prosecution (“appin cited documents”) and all documents cited or referenced in the appin cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy was created Mar. 30, 2023, is named J0178_02001SL.xml and is 168,237 bytes in size.

### FIELD OF THE INVENTION

The present invention relates to plants from the genus Alstroemeria L. with a new flower type.

### BACKGROUND OF THE INVENTION

Alstroemeria is a genus of flowering plants in the family Alstroemeriaceae. Alstroemeria plants are fairly easy to cultivate. The flowers are decorative and have a long vase life. They are commonly used as cut flowers but are also used in gardens.

A characteristic of Alstroemeria plants is that their blooms consist of umbels with a row of leaf-like, twisted bracts, and several branches with multiple flowers, which are attached to the stem through peduncles and pedicels. Furthermore, flowers in regular Alstroemeria plants contain an outer row of three tepals, and an inner row of three tepals.

In horticulture, there is always a desire to produce new varieties to add to the diversity in commercial cut flowers and garden plants.

Citation or identification of any document in this application is not an admission that such document is available as prior art to the present invention.

### SUMMARY OF THE INVENTION

In the research leading to the present invention an Alstroemeria plant with a novel flower type was developed. A characteristic in the new plants is the gradual transition from the leaves at the upper part of the shoots or stems to the leaf-like tepals from the single flower. The single large flower consists of a degenerated umbel, lacking peduncles and pedicels, and contains multiple, more than six, tepals. Some tepals are partially twisted. The fact that the Alstroemeria plant may comprise only one single flower per stem, which flower may comprise more than 6 tepals, is clearly different from existing Alstroemeria.

The invention thus relates to an Alstroemeria L. plant, which may comprise a single flower per stem, which flower may comprise more than 6 tepals. This new trait is called herein “double-type flower”.

The Alstroemeria flower of the invention may comprise more than 6 tepals per stem so that a flower has about 10 to 40 tepals, in particular 15 to 35 tepals, more in particular 20 to 30 tepals.

The tepals are arranged in a dense spiral structure with a gradual transition from leaf-like tepals to tepals, stamen-like tepals, stamens and pistil(s). The pistils are usually degenerated. The number of stamens is variable, ranging from absent to more than six as found in regular flowers. In some shoots of an individual plant, the spiral structure in the flowers is elongated, resulting in decorative foliage along the upper part of the shoots. Some varieties show more shoots with decorative foliage than others.

After propagation of the new plants, the continuous formation of shoots with a large single flower and shoots with decorative foliage is found to be a stable characteristic retained in successive growing seasons. If within a plant a shoot does carry a flower, it is a single, large, double-type flower according to the invention. It is therefore possible to obtain the new flower type during a production period of at least two years. Flowers are usually produced all year round. Production in areas with sufficient daylight is achieved without additional lighting, whereas in temperate zones artificial lighting is required.

The new flower type may comprise one flower that is larger than the individual flowers born in umbels in the regular Alstroemeria plants. In regular plants the flowers are usually up to 9 cm in diameter. According to the invention the diameter of the flowers is more than 10 cm.

The new flower type is the result of a dominant mutant gene. The trait of the invention is thus a dominant trait. The inheritance of the formation of double-type flowers occurs most efficiently through the male line.

The invention further relates to five QTLs which contribute to the double flower trait. These five QTLs are QTL_p_LG5, QTL_p_LG10, QTL_p_LG22_1, QTL_p_LG22_2, QTL_m_LG3. Plants having the maternal haplotype at four of the five QTLs have a “standard flower” phenotype, whereas plants having the paternal haplotype at at least one of QTLs QTL_p_LG5, QTL_p_LG10, QTL_p_LG22_1, QTL_m_LG3 have a “dual flower” phenotype.

The invention further relates to markers for the detection of the QTLs. The marker sequences of these markers are provided in Table 4b.

The invention relates in particular to the markers Contig15834c_1178 for QTL_p_LG5, Contig11637c_525 for QTL_p_LG10, Contig11429_3551_07 QTL_p_LG22_1, Contig27369sc_156_02 for QTL_p_LG22_2 and Contig9145_477_03 for QTL_m_LG3. The Alstroemeria L. plant of the invention may comprise no peduncles and no pedicels.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

The invention will be further illustrated in the example that follows which shows three new varieties, which are given for illustration purposes only and are not intended to limit the invention in any way. In this example reference is made to the following figures:

FIG. 1 Flower of mutant plant 02S1032-005.

FIG. 2A-2B Several examples of flowers and shoots from breeding program with plant 02S1032-005.

FIG. 3A Flower variety BE717.

FIG. 3B Side view flower variety BE717.

FIG. 4A Flower variety BE724.

FIG. 4B Side view flower variety BE724.

FIG. 5 Decorative foliage upper part shoots of variety BE724.

FIG. 6A Flower variety BK782.

FIG. 6B Side view flower variety BK782.

FIG. 7 Decorative foliage upper part shoots of variety BK782.

FIG. 8A Flower variety BE713.

FIG. 8B Side view of flower variety BE713.

FIG. 9 Decorative foliage upper part shoots of variety BE713.

FIG. 10—Overview of parental genotype dosage scores for SNP markers called in F1 population 20-0585.

FIG. 11—Histograms depicting the distribution of ordinal scores (left) or binary trait values (right) of flower type in F1 population 20-0585.

FIG. 12—Plot showing QTL obtained by mapping the “binary” double flower trait in F1 population 20-0585 on the paternal linkage map. Blue line indicates LOD scores according to the standard interval mapping model, red line indicates LOD scores according to the marker regression model. Dashed red and blue lines indicate the LOD threshold of both models respectively, as determined by performing a permutation test with 1000 permutations. Marker density along the 22 linkages groups is plotted on the X-axis.

FIG. 13—Plot showing QTL obtained by mapping the ordinal flower type trait in F1 population 20-0585 on the paternal linkage map. Blue line indicates LOD scores according to the standard interval mapping model, red line indicates LOD scores according to the marker regression model. Dashed red and blue lines indicate the LOD threshold of both models respectively, as determined by performing a permutation test with 1000 permutations. Marker density along the 22 linkages groups is plotted on the X-axis.

FIG. 14—Plot showing QTL obtained by mapping the “binary” double flower trait in F1 population 20-0585 on the maternal linkage map. Blue line indicates LOD scores according to the standard interval mapping model, red line indicates LOD scores according to the marker regression model. Dashed red and blue lines indicate the LOD threshold of both models respectively, as determined by performing a permutation test with 1000 permutations. Marker density along the 13 linkages groups is plotted on the X-axis.

FIG. 15—Plot showing QTL obtained by mapping the ordinal flower type trait in F1 population 20-0585 on the maternal linkage map. Blue line indicates LOD scores according to the standard interval mapping model, red line indicates LOD scores according to the marker regression model. Dashed red and blue lines indicate the LOD threshold of both models respectively, as determined by performing a permutation test with 1000 permutations. Marker density along the 22 linkages groups is plotted on the X-axis.

FIG. 16—Graphical representation of markers with LOD scores higher than 3.0 at the five detected QTLs for the Double Flower trait in population 20-0585. Markers are vertically ordered on map position. Maternal marker genotypes are coloured in blue, paternal marker genotypes are coloured in red, missing genotype scores are coloured in grey. F1 individuals are ordered on phenotypic value, with a separation between individuals with “Standard” flowers and individuals with “Dual” flowers.

FIG. 17—Boxplot of the “Flower type” ordinal values of population 20-0585; separated by genotype, at each of the five peak markers for the QTLs detected in population 20-0585.

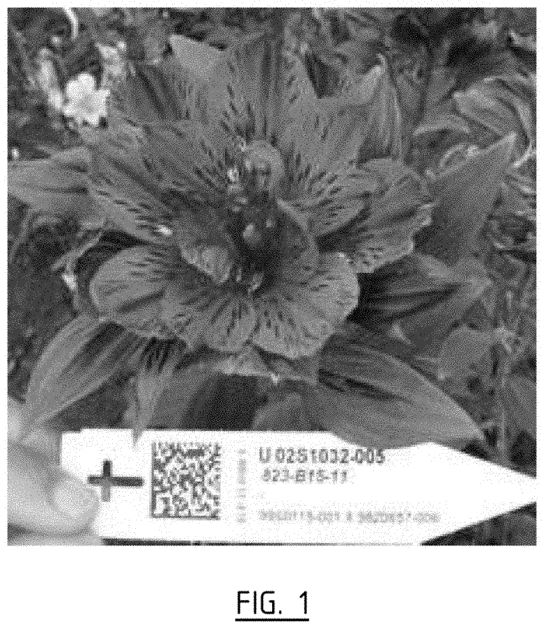

### DETAILED DESCRIPTION OF THE INVENTION

In the research leading to the present invention an Alstroemeria plant with a novel flower type was developed. A characteristic in the new plants is the gradual transition from the leaves at the upper part of the shoots or stems to the leaf-like tepals from the single flower. The single large flower consists of a degenerated umbel, lacking peduncles and pedicels, and contains multiple, more than six, tepals. Some tepals are partially twisted. The fact that the Alstroemeria plant may comprise only one single flower per stem, which flower may comprise more than 6 tepals, is clearly different from existing Alstroemeria.

The invention thus relates to an Alstroemeria L. plant, which may comprise a single flower per stem, which flower may comprise more than 6 tepals. This new trait is called herein “double-type flower”.

The Alstroemeria flower of the invention may comprise more than 6 tepals per stem so that a flower has about 10 to 40 tepals, in particular 15 to 35 tepals, more in particular 20 to 30 tepals.

The tepals are arranged in a dense spiral structure with a gradual transition from leaf-like tepals to tepals, stamen-like tepals, stamens and pistil(s). The pistils are usually degenerated. The number of stamens is variable, ranging from absent to more than six as found in regular flowers. In some shoots of an individual plant, the spiral structure in the flowers is elongated, resulting in decorative foliage along the upper part of the shoots. Some varieties show more shoots with decorative foliage than others.

After propagation of the new plants, the continuous formation of shoots with a large single flower and shoots with decorative foliage is found to be a stable characteristic retained in successive growing seasons. If within a plant a shoot does carry a flower, it is a single, large, double-type flower according to the invention. It is therefore possible to obtain the new flower type during a production period of at least two years. Flowers are usually produced all year round. Production in areas with sufficient daylight is achieved without additional lighting, whereas in temperate zones artificial lighting is required.

The new flower type may comprise one flower that is larger than the individual flowers born in umbels in the regular Alstroemeria plants. In regular plants the flowers are usually up to 9 cm in diameter. According to the invention the diameter of the flowers is more than 10 cm.

The new flower type is the result of a dominant mutant gene. The trait of the invention is thus a dominant trait. The inheritance of the formation of double-type flowers occurs most efficiently through the male line.

The Alstroemeria L. plant of the invention may comprise no peduncles and no pedicels.

Unlike the regular Alstroemeria flowers, which are slightly zygomorphic, the Alstroemeria flower of the invention is not zygomorphic. This means that the flower is not bilaterally symmetrical. In one embodiment, the flower is actinomorphic. This means that the flower is radially symmetrical. This production of actinomorphic flowers in a species that usually produces zygomorphic flowers is called pelorism.

In the Alstroemeria L. plant of the invention the tepals of the one flower per stem are arranged in a dense spiral structure at the top of the stem. This means that the placement of the tepals on the stem is not elongate but more flattened.

In one embodiment, a part of a leaf shows the flower colour. Some leaves on a stem, usually the ones closest to the flower, can have some colour. These leaves then have the colour of the tepals but not over the whole surface of a leaf but rather as streaks. The streaks are often located along the edges of the leaves and may cover only the edges of the leaf with normal green tissue in the middle of the leaf. Within a stem there can be a gradual transition from green leaves to leaves that partially show the flower colour to full coloured tepals.

In one embodiment, a stamen is fused together with a tepal and thus an integral part thereof

The ovary on the Alstroemeria L. plants of the invention is different from the ovary of regular Alstroemeria plants. In one embodiment, the ovary is rudimentary or malformed. In the plant of the invention, the pistils are usually degenerated. The female reproductive organs are thus non-functional. The plant of the invention is however fertile because of the presence of functioning stamens. An Alstroemeria plant of the invention can thus be used as the male parent in a cross. At least one copy of the gene causing the trait of the invention is mutant. In further embodiments, the plant may comprise two, three or four copies of the mutant trait. Since the trait is dominant, there will be progeny showing the trait even when the male parent carries only one mutant allele. Plants with the trait can be easily selected based on the phenotype.

In one embodiment, a stem may comprise 1 to 3 additional flowers. These flowers are not part of the main flower and are each located on a pedicel. These flowers are smaller than the main flower and comprise 6 tepals.

Seeds of plants of the invention showing the single double-type flower per stem are deposited at NCIMB Ferguson Building, Craibstone Estate, Bucksburn, Aberdeen AB21 9YA. The seeds of the deposit are a segregating population. To obtain plants producing the flowers of the invention, a selection step is required. This can be a visual selection.

In the present application the terms “Alstroemeria” and “Alstroemeria” are used interchangeably.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Genetics and Breeding History

In 2003 a whole plant mutant (called herein “mutant plant 02S1032-005”) was found in a population of seedlings. The mutant was used to develop the plant of the invention in a breeding program.

The tetraploid plants from this program are fertile, but since female reproductive organs are usually rudimentary or lacking in the flowers, they are best suitable to be used as male parents in crossings. The feature of the formation of double-type flowers seems to be a dominant trait, which can be observed in about 25-50% of the offspring from a crossing with an Alstroemeria female parent with regular type flowers.

In order to obtain varieties which can be commercialized, crossings are performed with diploid female parents and tetraploids male parent with the desired feature of doubleness. Their seedlings are triploid hybrids, having double-type flowers. Plants BE717, BE724 and BK782 are described below as an example.

### Example 2: Plant Material and Methods

The breeding process of Alstroemeria hybrid plants has been described by Aros et al. (2019) Molecular and morphological characterization of new interspecific hybrids of Alstroemeria originated from A. caryophylleae scented lines. Euphytica 215:93. Breeding and initial selection took place in greenhouses in Rijsenhout, the Netherlands.

Young plants carrying the invention are grown as described in the Alstroemeria cultivation brochure from Van Zanten Breeding B.V. (2016, available upon request).

Description of flowers of the invention was made in October 2021 in Rijsenhout, the Netherlands. These flowers were obtained by planting of in-vitro propagated plants in the last week of July in a greenhouse where 16 hrs of light is maintained with lamps (40 Lux). In July, the average day temperature was 22° C., and average night temperature 14° C. In October the average day temperature was 15° C. and the average night temperature 8° C.

Further selection took place in the plastic greenhouses at Colibri Farms, Via Subachoque 8-57, Madrid, Cundinamarca, Colombia. The average temperature there was 17° Celsius, average day temperature max 30° Celsius, and average night temperature 8-12° Celsius.

### Example 3: Flower Characteristics of the New Plants

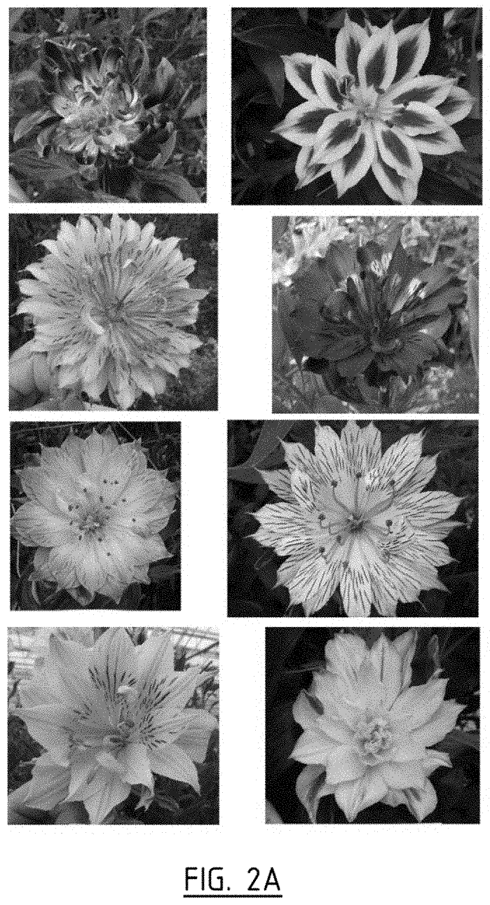

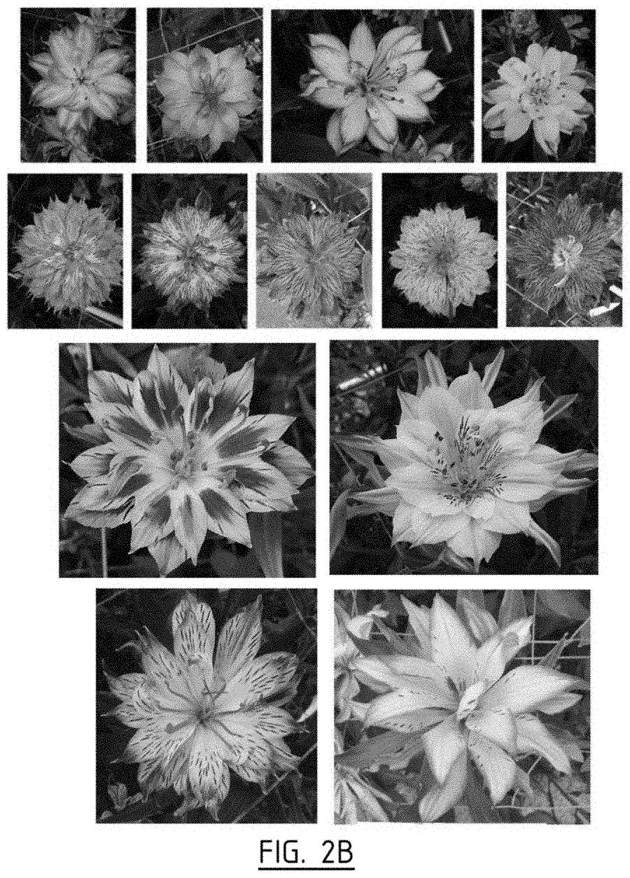

The colour range of the flowers of the new plants is similar to that of regular Alstroemeria plants. This is also the case for the flower pattern, including the presence of a yellow central zone and stripes in the tepals in some varieties. The size and colour of stamens and pistil, if present, are similar to those of regular Alstroemeria plants. Flowers of a number of new plants are illustrated in FIG. 2.

The number of tepals per flower in the new plants varies through the season but is always higher than 6.

The present invention will be further illustrated in the following varieties

In the following description, colour references are made to The Royal Horticultural Society Colour Chart, 2015 edition.

### Example 4: Variety BE717

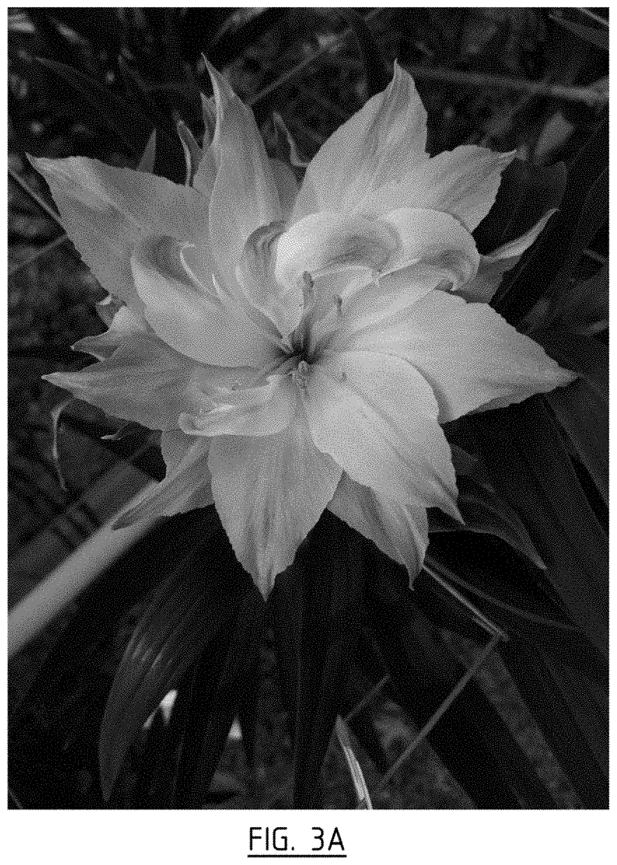

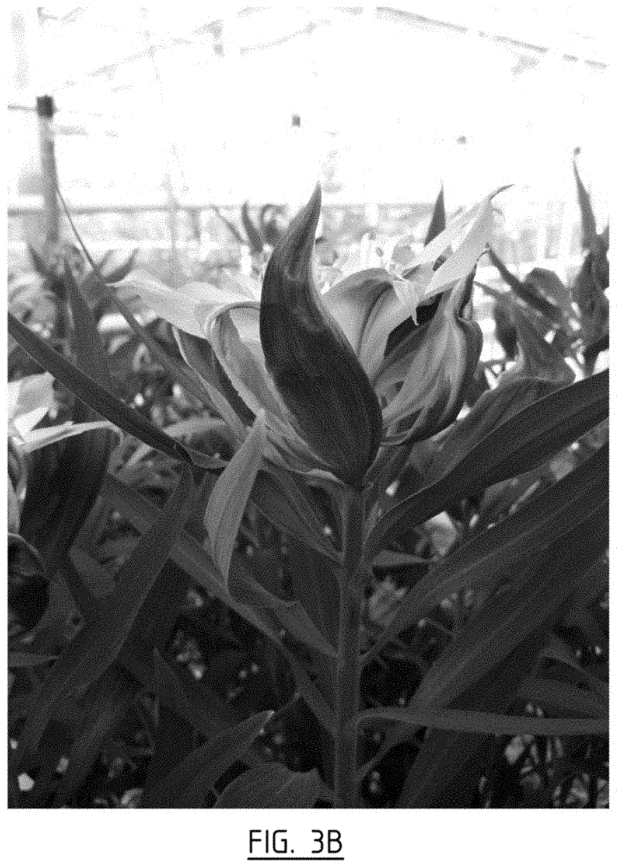

Flowers of variety BE717 are shown in FIGS. 3a and 3b. These flowers have the following characteristics:
Flower height: about 7 cm
Flower size: about 10 cm
Flower longevity on plant: at least 1.5 week
Number of tepals per flower: about 25 (first flush) to 15 (in later production)
Curvature tepals: straight, sometimes twisted
Length of tepals: about 7 cm in the outer rows to 4 cm in the inner rows
Width of tepals: about 3 cm in the outer rows to 2 cm in the inner rows
Shape of tepals: elliptical (outer rows) to oblanceolate (middle and inner rows)
Apex of tepals: acute to acuminate
Margin of tepals: entire to narrowly serrated
Texture: smooth
Lustre: matte
Colour pattern in tepals:
upper part Red-purple 70D, middle and basal part White 155A, and Green 138B in median zone (outer rows); upper part Red-purple 70D, middle and basal part White 155A (middle rows); upper part Red-purple 70D, middle part Yellow 3D and basal part White 155A (inner rows)
Numbers of stamens: 4 to 6
Length of filament: 6 cm
Width of filament: about 1 mm
Colour of filament: Red-purple 62D
Shape of anther: elliptical
Colour of anther: Greyed-Yellow 160D
Colour of pollen: Greyed-Orange 163D
Number of pistils: 1
Length of style and stigma: 5 cm
Colour of style and stigma: Red-purple 70C
Length of ovary: 4-5 mm
Width of ovary: 8-10 mm
Colour of ovary: Green 138A
Productivity (harvestable stems per m² per year, measured in Colombia): at least 280 for two years after planting
Production (measured in Colombia): stable formation of stems with single flowers (95-85%) and stems with decorative foliage (5-15%) for at least two years after planting
Vase life (after cool storage for 5 days): 20 — 28 days

### Example 5: Variety BE724

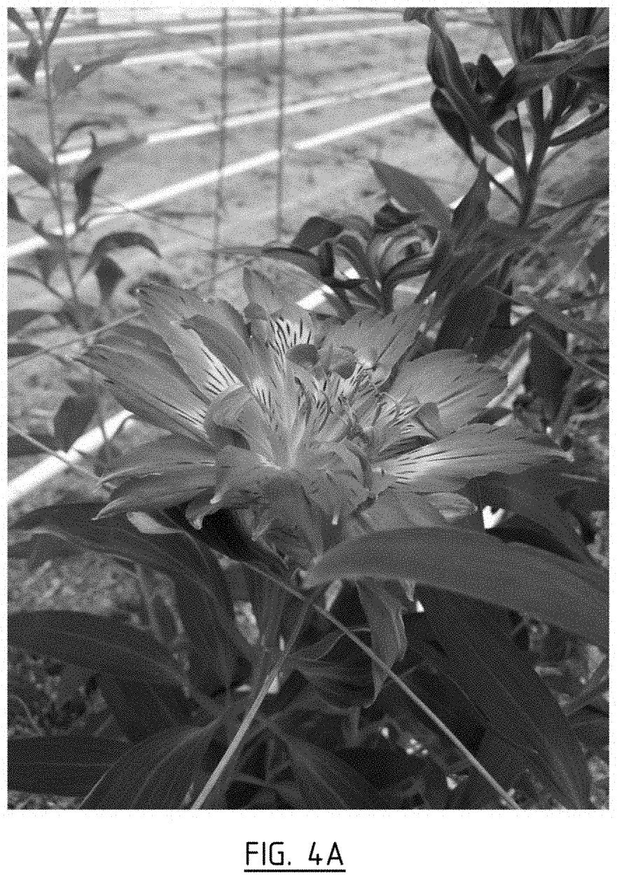

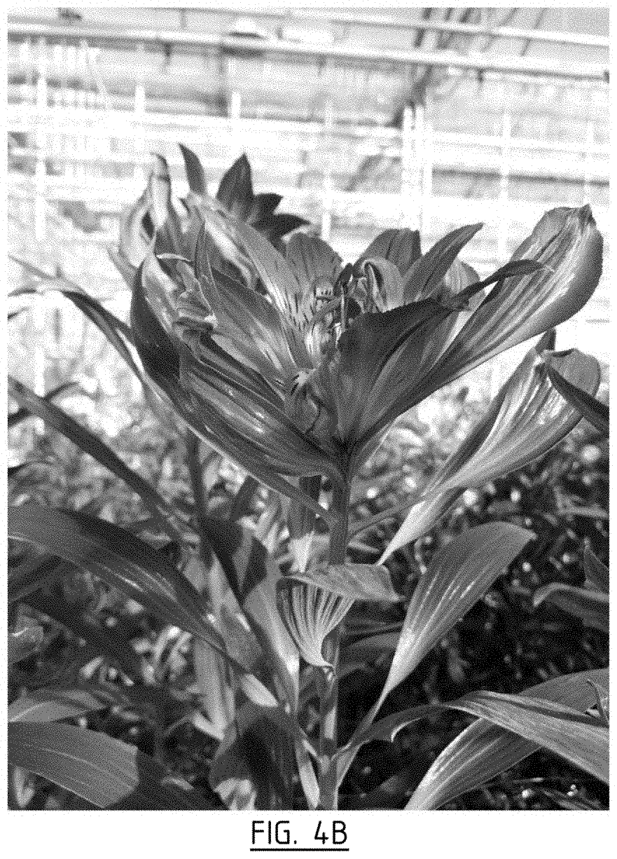

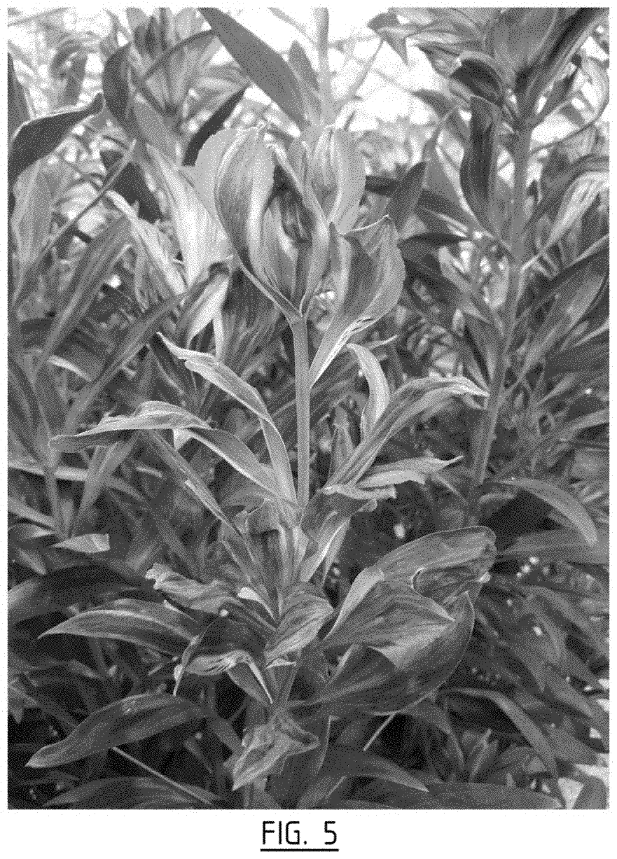

The flowers of variety BE724 are shown in FIGS. 4a and 4b, and decorative foliage in FIG. 5. These flowers have the following characteristics:
Flower height: about 7 cm
Flower size: about 14 cm
Flower longevity on plant: at least 1.5 week
Number of tepals per flower: 30 (first flush) to 20 (in later production) Curvature tepals: straight, sometimes twisted
Length of tepals: about 8 cm in the outer rows to 5 cm in the inner rows
Width of tepals: about 2 cm in the outer rows to 1.5 cm in the inner rows
Shape of tepals: elliptical and obovate (outer rows) to oblanceolate (inner rows)
Apex of tepals: acute to acuminate
Margin of tepals: entire to narrowly serrated
Texture: smooth
Lustre: glossy
Colour pattern in tepals:
upper part Red-purple 64B, middle and basal part Red-purple 62D, and Green 137A in median part (outer rows); upper part Red-purple 64B, middle part Yellow 12C, and basal part 62D (middle and inner rows)
Stripes in tepals: few (outer rows) to many (middle and inner rows)
Colour stripes: Greyed-Purple 185A
Numbers of stamens: 8 to 10
Length of filament: 4 cm
Width of filament: 1 mm
Colour of filament: Red-purple 64B
Shape of anther: elliptical
Colour of anther: Greyed Red-purple 187B
Colour of pollen: Greyed-yellow 161A
Number of pistils: 1-2
Length of style and stigma: 3.5 cm
Colour of style and stigma: Red-purple 64B
Length of ovary: 4-5 mm
Width of ovary: 8-10 mm
Colour of ovary: Green 138A
Productivity (harvestable stems per m² per year, measured in Colombia): at least 280 for two years after planting
Production (measured in Colombia): stable formation of stems with single flowers (95-85%) and stems with decorative foliage (5-15%) for at least two years after planting
Vase life (after cool storage for 5 days): 20-28 days

### Example 6: Variety BK782

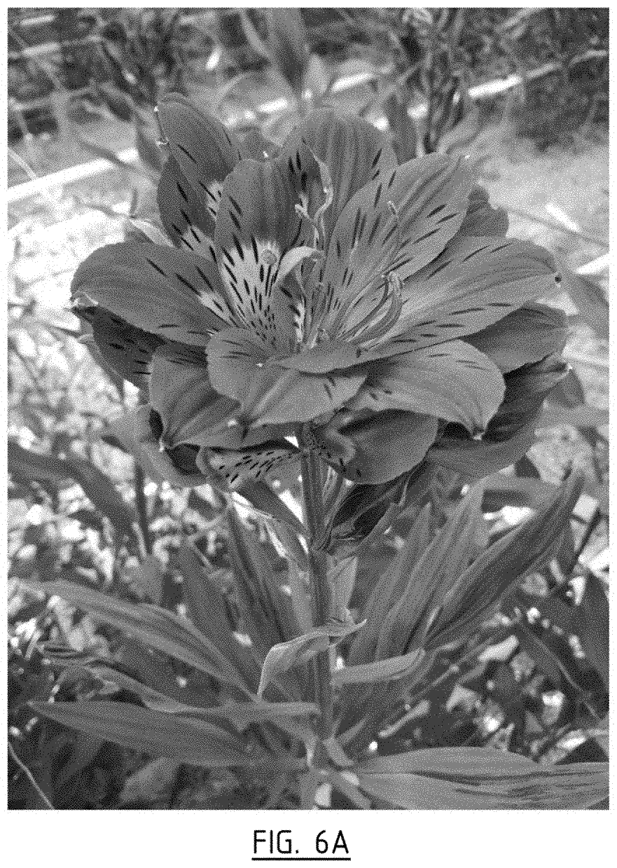

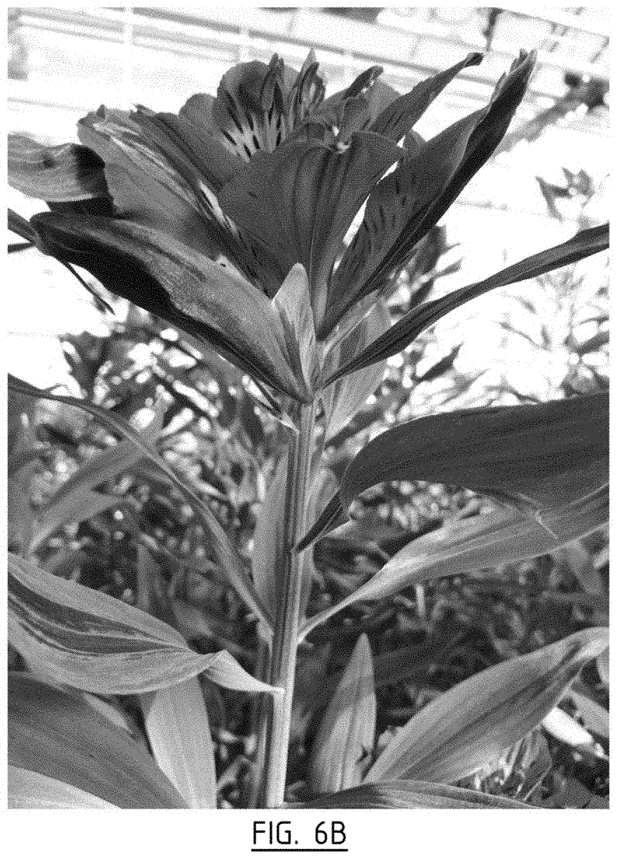

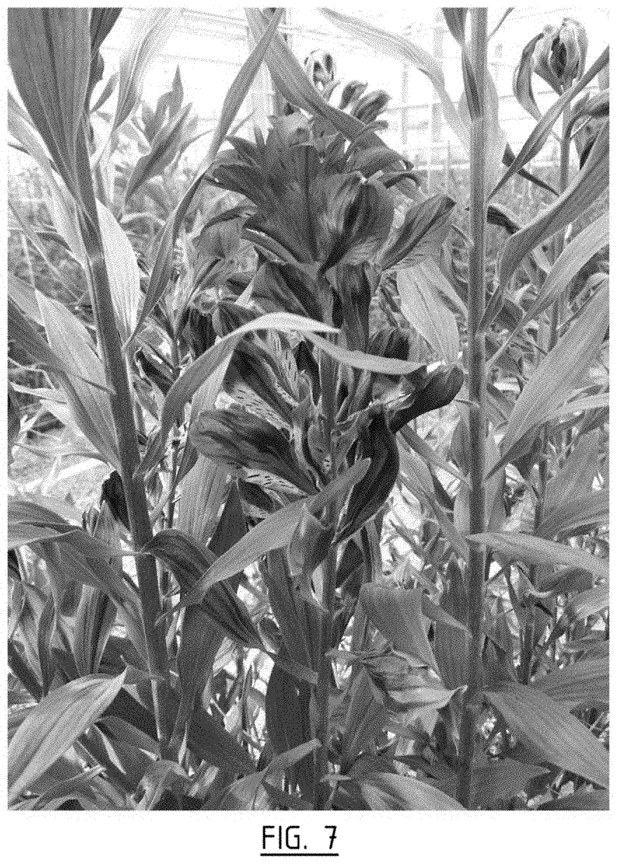

The flowers of variety BK782 are shown in FIGS. 6a and 6b, and decorative foliage in FIG. 7. These flowers have the following characteristics:
Flower height: about 8 cm
Flower size: 11 cm
Flower longevity on plant: at least 1.5 week
Number of tepals per flower: 25 (first flush) to 15 (in later production)
Curvature tepals: straight, sometimes twisted
Length of tepals: about 8 cm in the outer rows to 6 cm in the inner rows
Width of tepals: about 3 cm in the outer rows to 1.5 cm in the inner rows
Shape of tepals: elliptical and obovate (outer rows) to oblanceolate (inner rows)
Apex of tepals: Acute to acuminate
Margin of tepals: Entire to narrowly serrated
Texture: smooth
Lustre: glossy
Colour pattern in tepals:
lateral zones Red-purple N74A to D, median zone Green 138A and Greyed Purple 187A (outer rows); upper part Red-purple N74A, middle part Yellow-orange 21A, and basal part Red-purple N74D (middle rows); upper part Red-purple N74A, middle part Red 43D, and basal part Red-purple N74D (inner rows)
Stripes in tepals: few (outer rows) to many (middle and inner rows)
Colour stripes: Greyed-purple 187A
Numbers of stamens: 6-8
Length of filament: 6 cm
Width of filament: 1 mm
Shape of anther: elliptical
Colour of anther: Greyed-red 181A
Colour of pollen; Greyed-orange N167C
Number of pistils: 1
Length of style and stigma: 5 cm
Colour of style and stigma: Red-purple N74B
Length of ovary: 4-5 mm
Width of ovary: 8-10 mm
Colour of ovary: Green 138A with anthocyanin
Productivity (harvestable stems per m² per year, measured in Colombia): at least 280 for two years after planting
Production (measured in Colombia): stable formation of stems with single flowers (95-85%) and stems with decorative foliage (5-15%) for at least two years after planting
Vaselife (after cool storage for 5 days): 20-28 days

### Example 7: Variety BE713 7

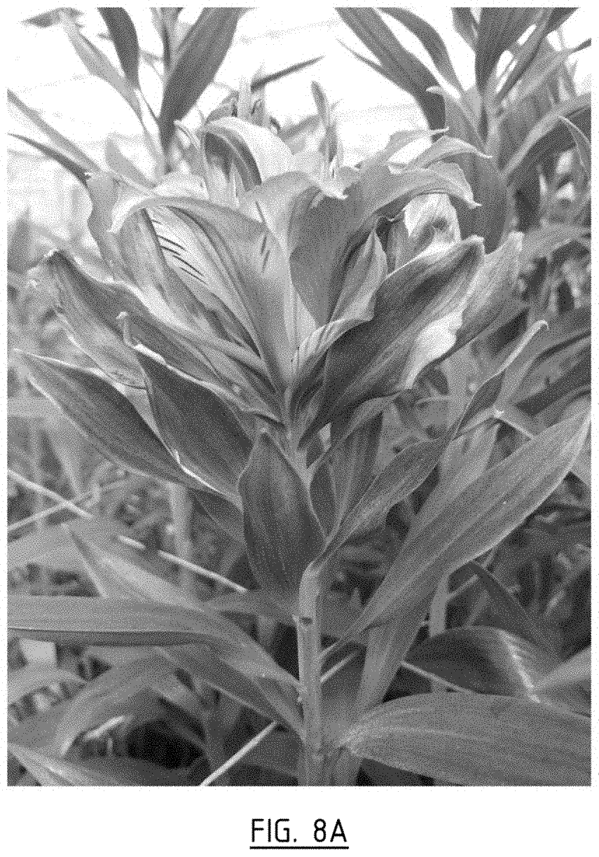

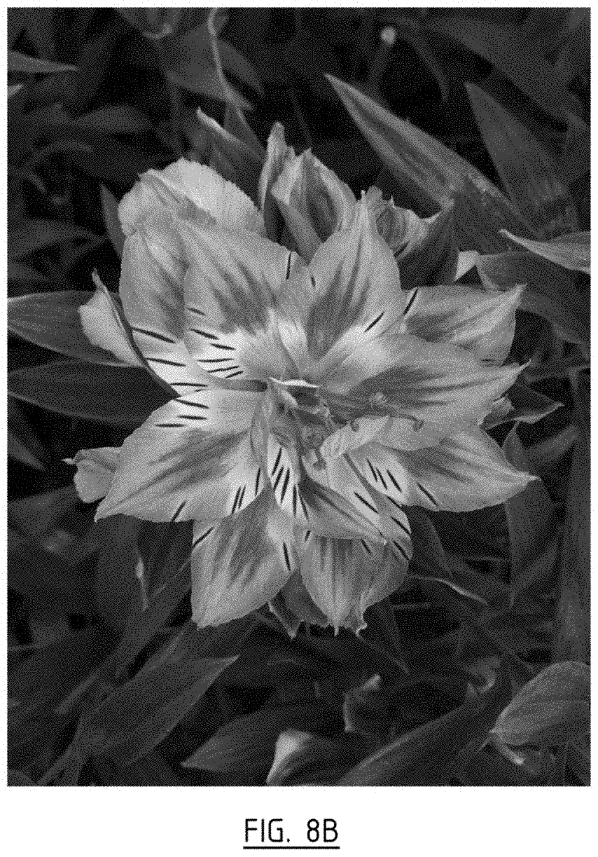

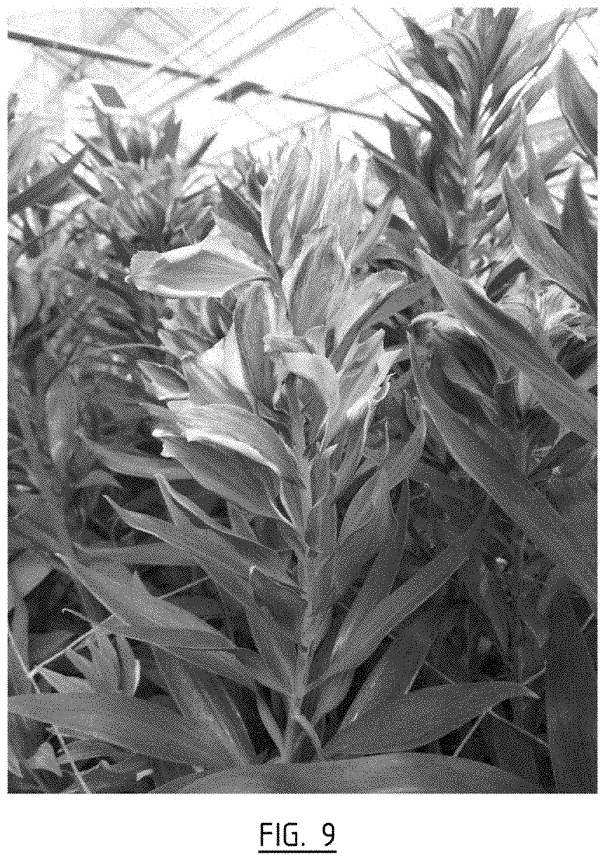

The flowers of variety BE713 are shown in FIGS. 8a and 8b, and decorative foliage in FIG. 9 These flowers have the following characteristics:
Flower height: about 6 cm
Flower size: about 10-11 cm
Flower longevity on plant: at least 1.5 week
Number of tepals per flower: 30 (first flush) to 20 (in later production) Curvature tepals: straight, sometimes twisted
Length of tepals: about 9 cm in the outer rows to 7 cm in the inner rows
Width of tepals: about 3 cm in the outer rows to 1.5 cm in the inner rows
Shape of tepals: elliptical and obovate (outer rows) to oblanceolate (inner rows)
Apex of tepals: acute to acuminate
Margin of tepals: entire to narrowly serrated
Texture: smooth
Lustre: glossy
Colour pattern in tepals:

outer parts Red-purple 72D, inner part Green 137C with anthocyanin (outer rows); upper part Red-purple 72D with blush of Red-purple 70A, central part Yellow 8D,basal part Red-purple 73D (middle and inner rows)
Stripes in tepals: few (outer rows) to medium numbers (middle and inner rows)
Colour stripes: Greyed-Purple 183A
Numbers of stamens: 6 to 8
Length of filament: 5 cm
Width of filament: 2 mm
Colour of filament: Red-purple 70B
Shape of anther: elliptical
Colour of anther: Greyed-white 156A to Orange-white 159A
Colour of pollen: Greyed-orange 177C
Number of pistils: 1-2 (partially fused)
Length of style and stigma: 3.5 cm
Colour of style and stigma: Red-purple 70B
Length of ovary: 3 mm
Width of ovary: 5 mm
Colour of ovary: Green 138C
Productivity (harvestable stems per m² per year, measured in Colombia): at least 280 for two years after planting
Production (measured in Colombia): stable formation of stems with single flowers (95-85%) and stems with decorative foliage (5-15%) for at least two years after planting
Vase life (after cool storage for 5 days): 20-28 days

### Example 6 WP1 Genetic Mapping of the Trait

1.1 Dosage Scoring for Segregating F1 Populations

60 DNA samples isolated from segregating F1 population “20-0585”, plus the parents of this population and 67 other accessions, were genotyped using a proprietary Affymetrix “Axiom_WagAhRvZ” array developed for Van Zanten Holding B.V.

As the parents from the F1 population have a tetraploid genome, genotype dosages of markers can vary between 0 (homozygous for the reference allele) to 4 (homozygous for the alternative allele), with heterozygous dosages of 1, 2 or 3 in between. Axiom intensities were used to score genotype dosages in the population, using the R package FitPoly (v 3.0.0). Dosage scoring resulted in 19.210 markers successfully called for population 20-0585.

In majority, the called markers belonged to the simplex-nulliplex class (i.e. one parent has a genotype dosage of 1; whereas the other parent has a genotype dosage of 0) or the simplex-simplex class (i.e. both parents have a genotype dosage of 1).

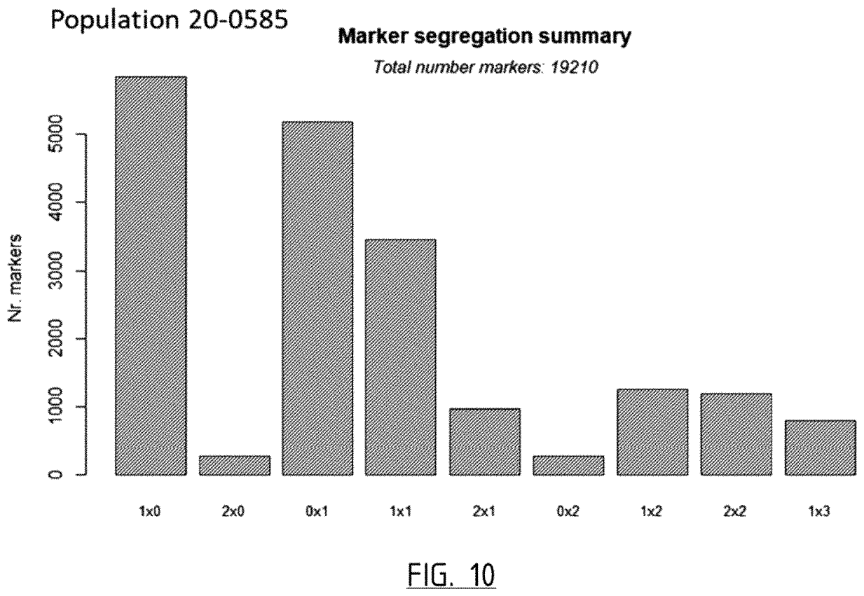

FIG. 10 gives an overview of the marker segregation summary.

During genotyping, it was discovered that in the population several F1 individuals had identical genotypes for all markers. This might be the result of e.g. one plant accidentally sampled twice. Especially since some of these identical genotypes had different phenotypes. Table 1 gives an overview of all genetically identical individuals. Duplicate genotypes were removed from further QTL analysis.

TABLE 1
Overview of detected duplicate genotypes

| Individual 1 | Individual 2 |
|---|---|
| 20-0585-011 | 20-0585-012 |
| 20-0585-014 | 20-0585-016 |
| 20-0585-034 | 20-0585-035 |

1.2 Linkage Map Construction

Diploid linkage mapping software CarteBlanche (v 1.1.0) was used to generate linkage maps of simplex-nulliplex markers, which segregate in F1 populations in a 1:1 ratio. This resulted in two linkage maps, one containing markers for which the mother has a dosage of 1 and the father a dosage of 0; and one for which the father has a dosage of 1 and the mother a dosage of 0. This resulted in two linkage maps consisting of 12 and 22 linkage groups, respectively.

1.3 QTL Mapping

The flowertype of individuals in the population were scored by Van Zanten Holding B.V. on an ordinal scale from 2 to 7. Table 2 gives an overview of the flowertype ordinal scale. It is assumed that the trait to produce “double flowers” is monogenic. Variations within the phenotype of plants with the “double flower” trait, ranging from “semi-sphere” to “true dual flowers”, are thought to be the result from a combination of genetic effects and environmental effects. Therefore, the phenotypes were also translated into a binary trait, i.e. “standard flowers” being all individuals with a score of 2, and “dual flowers” being all individuals with scores higher than 2.

TABLE 2
Overview of ordinal flowertype categories

| Score | Flowertype |
|---|---|
| 2 | Standard |
| 3 | Semi-sphere |
| 4 | Sphere |
| 5 | Sphere-Dual |
| 6 | Dual, irregular |
| 7 | True Dual |

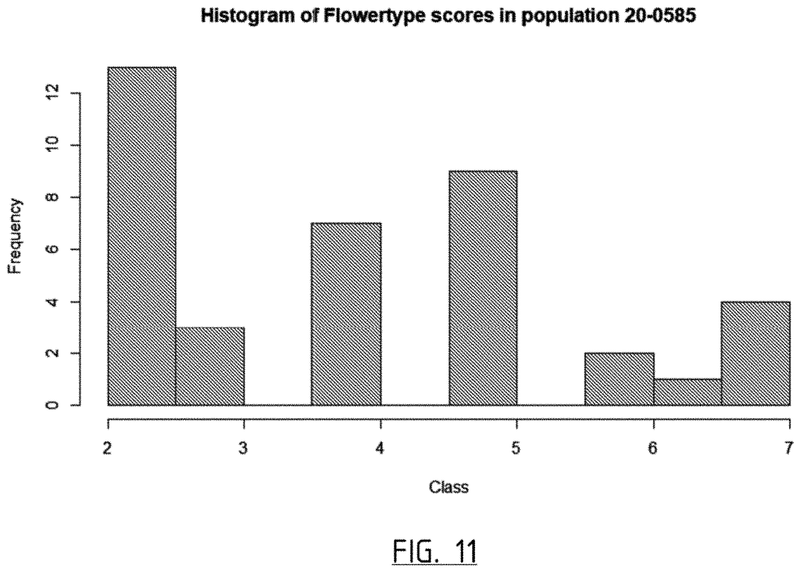

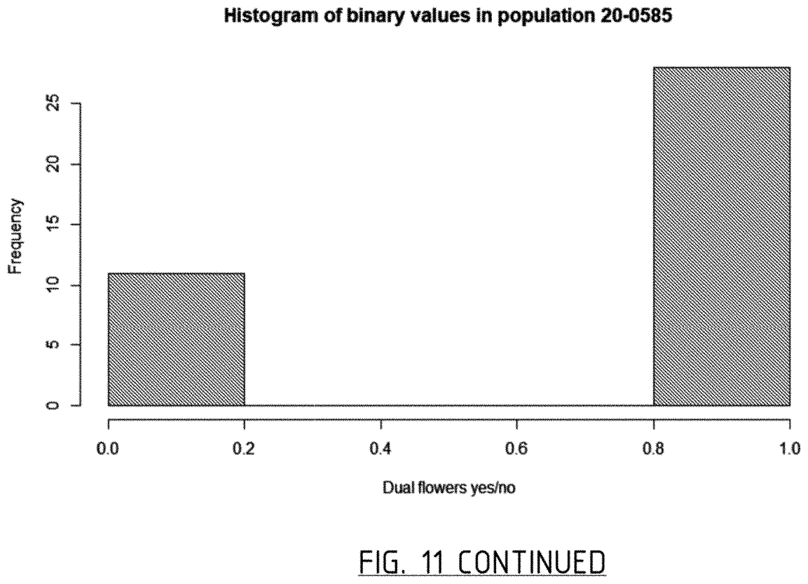

FIG. 11 show the distribution of flower types within population 20-0585 in “ordinal” and “binary” representation. It is visible that more than 50% of the populations have the double flower phenotype, indicating that it is statistically unlikely that the donor of the trait carries only one copy of the “double flower” allele (Chi-square test, p=0.006).

QTL mapping was performed using both the “ordinal” 2-7 phenotypes, as well as the “binary” dual/non-dual phenotypes, on each of the two linkage maps described in 1.2. Software package R/QTL (v 1.52) was used for QTL mapping, using both the “standard interval mapping” (EM) and the “marker regression” (MR) method. A permutation test consisting of 1000 permutations was performed to define the threshold for statistical significance.

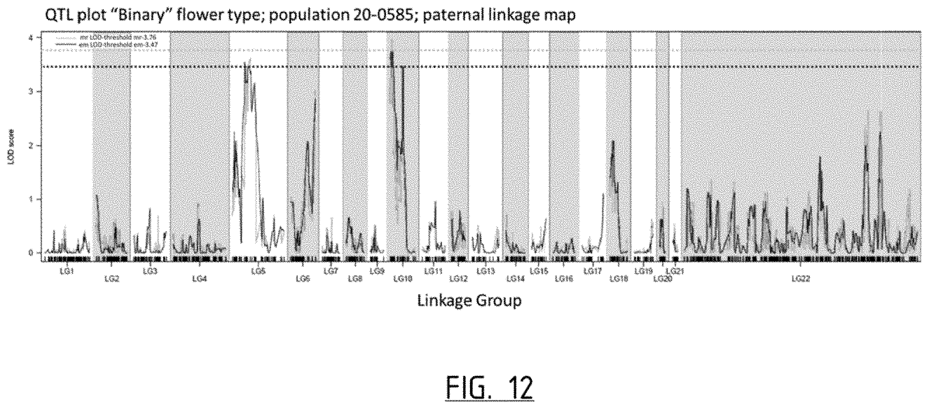

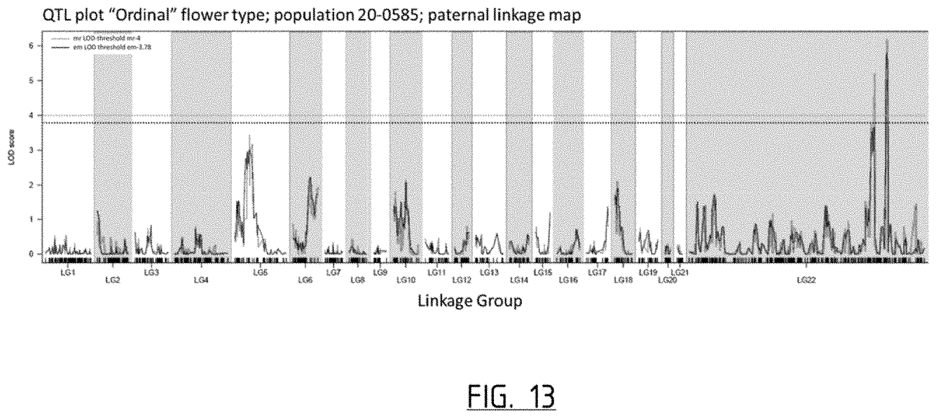

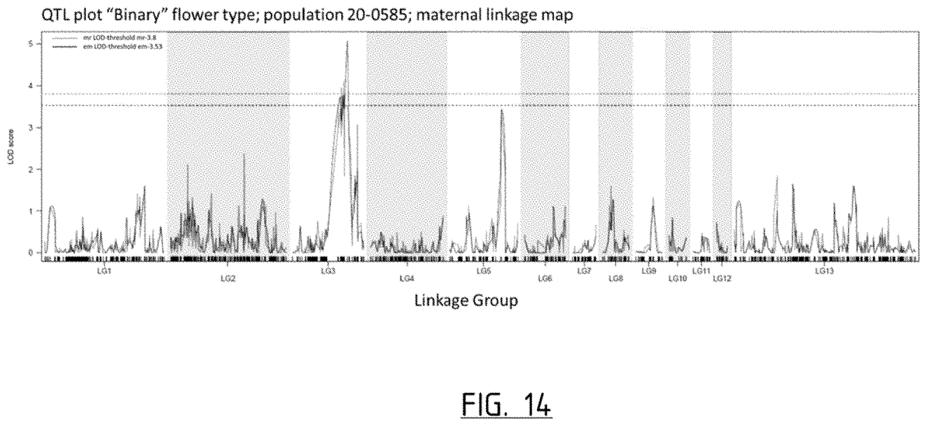

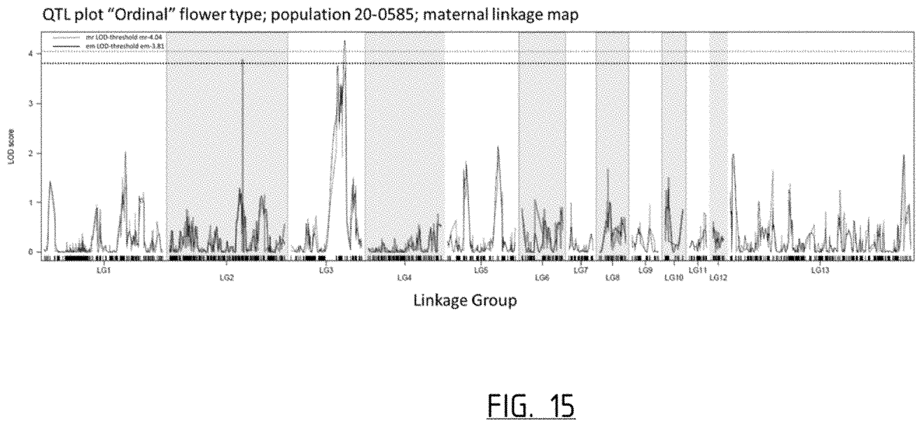

In population 20-0585; five significant QTLs were detected; four of which were found on the paternal linkage map, and one on the maternal linkage map. FIGS. 12 to 15 show the four QTL plots; and Table 3 provides information of the identified QTLs.

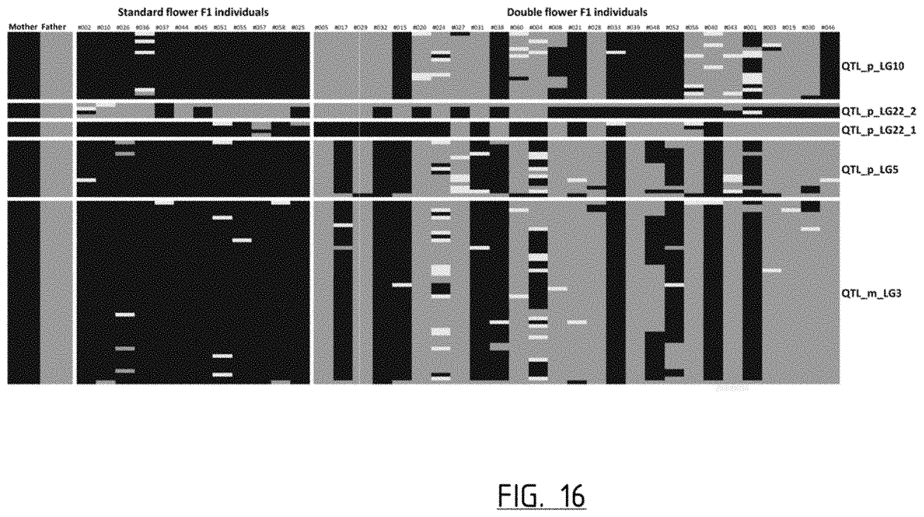

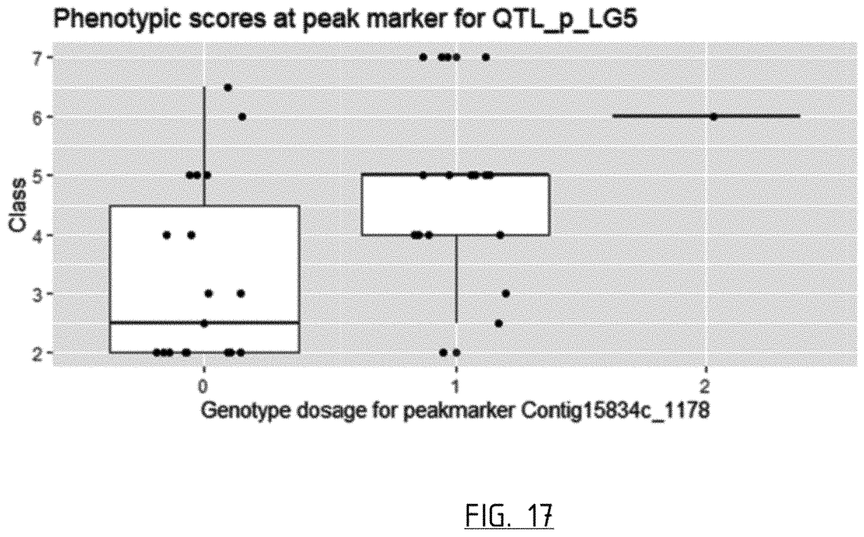

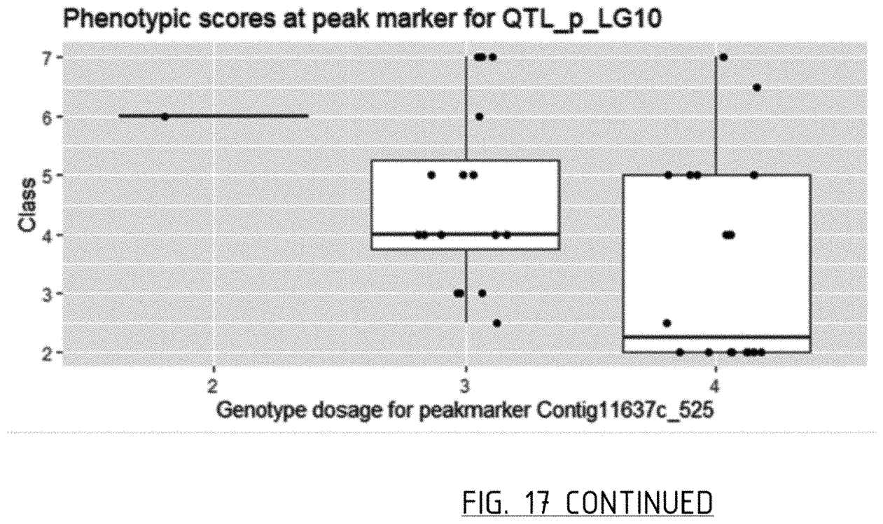

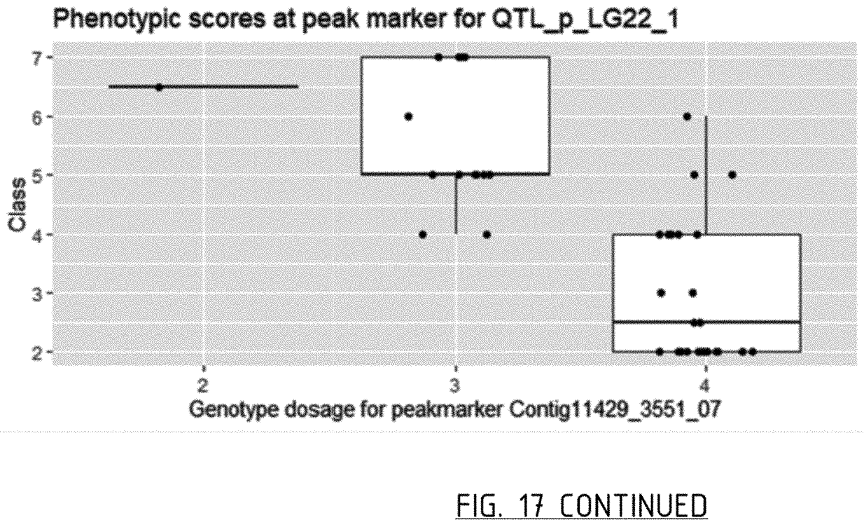

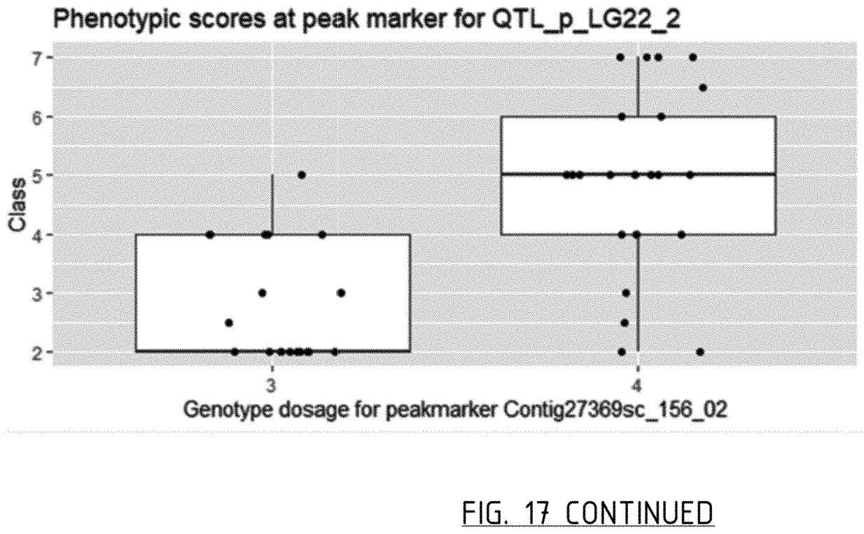

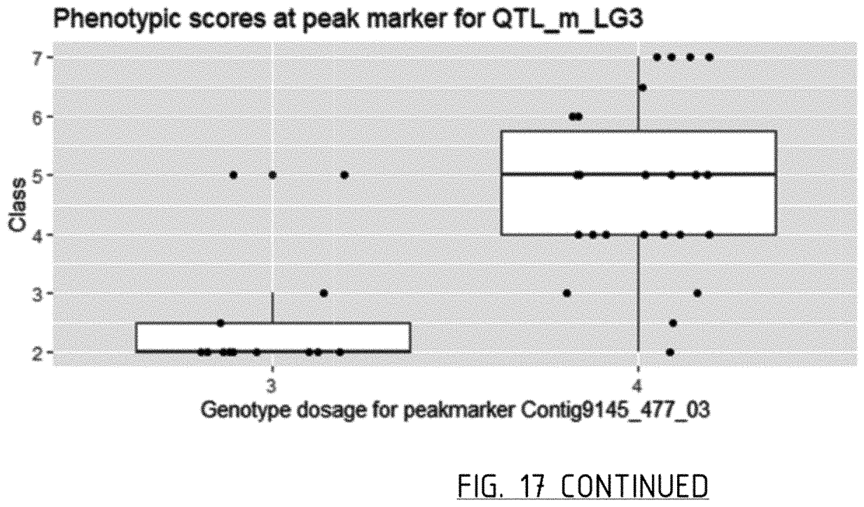

FIG. 16 gives a graphical representation of all markers with LOD scores>3.0, ordered along their genetic position (in centiMorgan) on linkage groups 3, 5, 10 and 22. It is visible from this figure that all F1 individuals with the “Standard flower” phenotype have the maternal haplotype at four of the five QTLs, the exception being QTL_p_LG22_2, at which the “Standard flower” F1 individuals in majority have the paternal haplotype. The F1 individuals with the “Dual flower” phenotype have the paternal haplotype at at least one of the five QTLs. In FIG. 17 the ordinal Flower Type scores of population 20-0585 are plotted, grouped by the genotypic scores at the five QTL peak markers.

TABLE 3
Overview of QTLs for the “Double Flower” trait detected in population 20-
0585. Names of peak markers are given, the position of the peak marker (paternal
or maternal linkage map, linkage group and position in centiMorgan), as well as
the LOD scores according to the interval mapping (IM) and marker regression (MR) models.

| | | | | | LOD | LOD |
|---|---|---|---|---|---|---|
| | | Linkage | Linkage | Position | score | score |
| QTL | Peak marker | map | group | (cM) | (IM) | (MR) |
| QTL_p_LG5 | Contig15834c_1178 | Paternal | LG5 | 43.5 | 3.55 | 1.99 |
| QTL_p_LG10 | Contig11637c_525 | Paternal | LG10 | 1.4 | 3.76 | 3.95 |
| QTL_p_LG22_1 | Contig11429_3551_07 | Paternal | LG22 | 719.3 | 5.81 | 6.17 |
| QTL_p_LG22_2 | Contig27369sc_156_02 | Paternal | LG22 | 672.5 | 3.68 | 4.15 |
| QTL_m_LG3 | Contig9145_477_03 | Maternal | LG3 | 197.8 | 5.08 | 4.10 |

Genotyping of the segregating F1 population 20-0585 by means of a 60k Axiom array allowed estimating genotype dosages of ˜19k informative markers. The majority of called markers were either simplex-nulliplex or simplex-simplex segregating SNPs, which have relatively straightforward expected segregation ratios (1:1 and 1:2:1, respectively).

Two parent-specific linkage maps were generated, based on simplex-nulliplex markers, using mapping software dedicated for diploid populations.

Since the double flower mutant is the result of a spontaneous mutation, it is expected to be a monogenic trait. The segregation ratios between standard and double flowers observed in both F1 populations are skewed from the 1:1 ratio which would have been expected if the donor (i.e. the father) of the trait would carry one “double flower” allele. It is therefore likely that the causal allele is present in a higher dosage.

QTL mapping in population 20-0585 resulted in the identification of five QTLs which contribute to the double flower trait. It should be noted that two QTLs were only found using the ordinal flower type classes (i.e. QTL_p_LG22_1 and QTL_p_LG22_2); whereas two others (i.e. QTL_p_LG5 and QTL_p_LG10) were only found using the “binary” recoding of the double flower trait. The fifth QTL (QTL_m_LG3), mapped on the maternal linkage map, was found using both approaches. It should further be noted that the names of markers in QTL_p_LG5 and QTL_m_LG3 indicate that the markers are for a large part of the same physical “contigs”. In other words, QTL_p_LG5 and QTL_m_LG3 are presumably a paternal and a maternal haplotype of the same QTL. The other three detected QTLs, QTL_p_LG10, QTL_p_LG22_1 and QTL_p_LG22_2 could either be other paternal haplotypes at the same locus, or alternatively one or more of them could represent a second causal gene at a different locus.

A graphical representation of the haplotypes of the population at the five QTL regions indicates that F1 individuals with a “standard” flowertype have the maternal haplotype at four of the five loci. Having at least one paternal haplotype (with the exception of QTL_p_LG22_2) generally leads to a “double flower” trait, indicative of a dominant mutation.

In population 20-0585 the “double flower” trait was successfully mapped to five QTLs. Plants having the maternal haplotype at four of the five QTLs are expected to have a “standard flower” phenotype, whereas plants having the paternal haplotype at at least one of four QTLs (QTL_p_LG22_2 excluded) are expected to have a “dual flower” phenotype.

Tables 4a and 4b show the data for all described markers. Table 4a shows the marker name, LG, Position in cM, LOD scores, QTL names, whether the marker is a peak marker or a surrounding marker and the dose of the marker in genotype 16_0700_003 (mother) and genotype 16_3247_001 (father). Table 4b shows the marker sequences. Peak markers are highlighted.

TABLE 4a

| | | | | | | geno- | geno- |
|---|---|---|---|---|---|---|---|
| | | | | | Peak_Sur- | type_16_0700_003 | type_16_3247_001 |
| Marker name | LG | Pos(cM) | LOD | QTL | rounding | (mother) | (father) |
| Contig4023_838_04 | LG3 | 164.400129 | 3.498900584 | QTL_m_LG3 | surrounding | 3 | 4 |
| Contig4014_1233_all | LG3 | 164.40013 | 3.498900476 | QTL_m_LG3 | surrounding | 1 | 0 |
| Contig23581c_155_all | LG3 | 165.700131 | 3.493418106 | QTL_m_LG3 | surrounding | 3 | 4 |
| Contig21666c_2330_all | LG3 | 171.600132 | 3.730327759 | QTL_m_LG3 | surrounding | 1 | 0 |
| Contig4435_509_02 | LG3 | 173.400133 | 3.453101231 | QTL_m_LG3 | surrounding | 3 | 4 |
| Contig33097c_465_03 | LG3 | 173.400134 | 3.453101243 | QTL_m_LG3 | surrounding | 3 | 4 |
| Contig48792c_899_all | LG3 | 173.500135 | 3.452803052 | QTL_m_LG3 | surrounding | 3 | 4 |
| Contig981_343_all | LG3 | 174.200136 | 3.162136013 | QTL_m_LG3 | surrounding | 0 | 1 |
| Contig11367_195_all | LG3 | 174.200137 | 3.162135445 | QTL_m_LG3 | surrounding | 2 | 1 |
| Contig48792c_1070_all | LG3 | 174.300138 | 3.161398193 | QTL_m_LG3 | surrounding | 1 | 0 |
| Contig3277c_1388_all | LG3 | 175.100139 | 3.161529924 | QTL_m_LG3 | surrounding | 1 | 0 |
| Contig11042_1125_all | LG3 | 175.10014 | 3.161529925 | QTL_m_LG3 | surrounding | 3 | 4 |
| Contig4435_448_05 | LG3 | 175.200141 | 3.162400703 | QTL_m_LG3 | surrounding | 4 | 3 |
| Contig38099c_803_all | LG3 | 176.300142 | 3.161530009 | QTL_m_LG3 | surrounding | 3 | 4 |
| Contig15039c_854_all | LG3 | 176.400143 | 3.161530026 | QTL_m_LG3 | surrounding | 1 | 0 |
| Contig11042_2095_12 | LG3 | 176.500144 | 3.161535435 | QTL_m_LG3 | surrounding | 3 | 4 |
| Contig2023_279_all | LG3 | 176.500145 | 3.16153544 | QTL_m_LG3 | surrounding | 3 | 4 |
| Contig12710c_2266_all | LG3 | 177.200146 | 3.452669442 | QTL_m_LG3 | surrounding | 1 | 0 |
| Contig2745_618_all | LG3 | 177.300147 | 3.452965468 | QTL_m_LG3 | surrounding | 1 | 0 |
| Contig12710c_1822_05 | LG3 | 177.300148 | 3.452965468 | QTL_m_LG3 | surrounding | 1 | 0 |
| Contig10052_518_04 | LG3 | 177.600149 | 3.452916322 | QTL_m_LG3 | surrounding | 3 | 4 |
| Contig4882_2237_all | LG3 | 177.80015 | 3.449323551 | QTL_m_LG3 | surrounding | 1 | 0 |
| Contig4972_2787_all | LG3 | 177.900151 | 3.224123633 | QTL_m_LG3 | surrounding | 1 | 0 |
| Contig4972_3573_all | LG3 | 177.900152 | 3.224123604 | QTL_m_LG3 | surrounding | 3 | 4 |
| Contig19967c_129_all | LG3 | 178.000153 | 3.2246831 | QTL_m_LG3 | surrounding | 3 | 4 |
| Contig283sc_148_04 | LG3 | 178.500154 | 3.452384196 | QTL_m_LG3 | surrounding | 3 | 4 |
| Contig21666c_4757_all | LG3 | 178.500155 | 3.452384556 | QTL_m_LG3 | surrounding | 1 | 0 |
| Contig9996_428_02 | LG3 | 178.700156 | 3.453133331 | QTL_m_LG3 | surrounding | 3 | 4 |
| Contig9550_487_03 | LG3 | 178.800157 | 3.45421814 | QTL_m_LG3 | surrounding | 1 | 0 |
| Contig21666c_4350_all | LG3 | 179.200158 | 3.762087453 | QTL_m_LG3 | surrounding | 1 | 0 |
| Contig23932c_1258_04 | LG3 | 179.200159 | 3.762087458 | QTL_m_LG3 | surrounding | 1 | 0 |
| Contig4797_1761_05 | LG3 | 179.30016 | 3.761998666 | QTL_m_LG3 | surrounding | 0 | 1 |
| Contig4794_1130_03 | LG3 | 179.600161 | 3.761735273 | QTL_m_LG3 | surrounding | 3 | 4 |
| Contig12710c_1627_all | LG3 | 179.600162 | 3.761735279 | QTL_m_LG3 | surrounding | 3 | 4 |
| Contig12710c_2551_02 | LG3 | 180.000163 | 3.763860716 | QTL_m_LG3 | surrounding | 1 | 0 |
| Contig14450sc_358_02 | LG3 | 181.900164 | 3.453352258 | QTL_m_LG3 | surrounding | 1 | 0 |
| Contig1477sc_165_03 | LG3 | 182.000165 | 3.453124211 | QTL_m_LG3 | surrounding | 1 | 0 |
| Contig10987_638_03 | LG3 | 182.300166 | 3.453752213 | QTL_m_LG3 | surrounding | 3 | 4 |
| Contig11321c_252_03 | LG3 | 184.400167 | 3.773978972 | QTL_m_LG3 | surrounding | 3 | 4 |
| Contig10685_81_05 | LG3 | 185.400168 | 3.765462125 | QTL_m_LG3 | surrounding | 1 | 0 |
| Contig5429_1938_all | LG3 | 185.400169 | 3.765461164 | QTL_m_LG3 | surrounding | 1 | 0 |
| Contig9806sc_215_12 | LG3 | 185.50017 | 3.764038025 | QTL_m_LG3 | surrounding | 1 | 0 |
| Contig1858_2184_all | LG3 | 185.500171 | 3.764037578 | QTL_m_LG3 | surrounding | 0 | 1 |
| Contig1858_1186_all | LG3 | 186.000172 | 3.459964392 | QTL_m_LG3 | surrounding | 3 | 4 |
| Contig5429_1938_all | LG3 | 186.800173 | 3.452920696 | QTL_m_LG3 | surrounding | 1 | 0 |
| Contig47217c_830_all | LG3 | 186.800174 | 3.452920671 | QTL_m_LG3 | surrounding | 1 | 0 |
| Contig10987_572_all | LG3 | 186.900175 | 3.450592275 | QTL_m_LG3 | surrounding | 0 | 1 |
| Contig1858_1910_all | LG3 | 186.900176 | 3.450592293 | QTL_m_LG3 | surrounding | 1 | 0 |
| Contig9145_477_03 | LG3 | 197.800177 | 5.080857952 | QTL_m_LG3 | Peak_marker | 3 | 4 |
| Contig25540c_69_06 | LG10 | 0 | 3.592996976 | QTL_p_LG10 | surrounding | 0 | 1 |
| Contig631_1175_03 | LG10 | 1.400001 | 3.764484887 | QTL_p_LG10 | surrounding | 0 | 1 |
| Contig11637c_525_all | LG10 | 1.400002 | 3.764484888 | QTL_p_LG10 | Peak_marker | 4 | 3 |
| Contig38500c_86_03 | LG10 | 1.500003 | 3.763059967 | QTL_p_LG10 | surrounding | 4 | 3 |
| Contig669_218_04 | LG10 | 1.700004 | 3.487031356 | QTL_p_LG10 | surrounding | 0 | 1 |
| Contig3891_979_04 | LG10 | 3.400005 | 3.451401015 | QTL_p_LG10 | surrounding | 0 | 1 |
| Contig5523_3142_03 | LG10 | 3.400006 | 3.451401012 | QTL_p_LG10 | surrounding | 4 | 3 |
| Contig5523_2455_04 | LG10 | 3.900007 | 3.452749037 | QTL_p_LG10 | surrounding | 4 | 3 |
| Contig11449_1176_all | LG10 | 4.000008 | 3.453111649 | QTL_p_LG10 | surrounding | 0 | 1 |
| Contig5523_3453_03 | LG10 | 4.100009 | 3.453115364 | QTL_p_LG10 | surrounding | 0 | 1 |
| Contig47887c_978_06 | LG10 | 4.10001 | 3.453115364 | QTL_p_LG10 | surrounding | 4 | 3 |
| Contig11054c_440_all | LG10 | 4.800011 | 3.45549306 | QTL_p_LG10 | surrounding | 4 | 3 |
| Contig29740c_640_04 | LG10 | 6.100012 | 3.763964301 | QTL_p_LG10 | surrounding | 4 | 3 |
| Contig12758c_410_04 | LG10 | 6.100013 | 3.763964306 | QTL_p_LG10 | surrounding | 4 | 3 |
| Contig8038_998_all | LG10 | 7.500014 | 3.712663757 | QTL_p_LG10 | surrounding | 4 | 3 |
| Contig9761_813_all | LG10 | 10.500015 | 3.27961138 | QTL_p_LG10 | surrounding | 4 | 3 |
| Contig2582c_1172_all | LG10 | 12.100016 | 3.2733047 | QTL_p_LG10 | surrounding | 4 | 3 |
| Contig19739c_664_02 | LG10 | 12.500017 | 3.027949907 | QTL_p_LG10 | surrounding | 4 | 3 |
| Contig1813sc_151_07 | LG22 | 714.900492 | 3.852791145 | QTL_p_LG22_1 | surrounding | 0 | 1 |
| Contig26701c_392_07 | LG22 | 717.600493 | 5.705192189 | QTL_p_LG22_1 | surrounding | 4 | 3 |
| Contig11429_3551_07 | LG22 | 719.300494 | 5.80523585 | QTL_p_LG22_1 | Peak_marker | 4 | 3 |
| Contig2648_3441_all | LG22 | 719.700495 | 5.781507143 | QTL_p_LG22_1 | surrounding | 0 | 1 |
| Contig50234c_876_05 | LG22 | 663.400432 | 3.418438717 | QTL_p_LG22_2 | surrounding | 4 | 1 |
| Contig9319_471_23 | LG22 | 669.500433 | 2.957744415 | QTL_p_LG22_2 | surrounding | 4 | 3 |
| Contig37704c_728_07 | LG22 | 670.800434 | 3.633644633 | QTL_p_LG22_2 | surrounding | 0 | 1 |
| Contig27369sc_156_02 | LG22 | 672.500435 | 3.67800901 | QTL_p_LG22_2 | Peak_marker | 4 | 3 |
| Contig15834c_1178_all | LG5 | 43.500048 | 3.55165266 | QTL_p_LG5 | Peak_marker | 0 | 1 |
| Contig1858_2184_all | LG5 | 46.700049 | 3.449181715 | QTL_p_LG5 | surrounding | 0 | 1 |
| Contig10987_572_all | LG5 | 48.10005 | 3.161840568 | QTL_p_LG5 | surrounding | 0 | 1 |
| Contig32205c_428_02 | LG5 | 48.200051 | 3.1614933 | QTL_p_LG5 | surrounding | 4 | 3 |
| Contig3804_255_04 | LG5 | 50.500052 | 3.161544162 | QTL_p_LG5 | surrounding | 0 | 1 |
| Contig9821_1567_all | LG5 | 50.600053 | 3.161608351 | QTL_p_LG5 | surrounding | 4 | 3 |
| Contig21666c_1131_07 | LG5 | 53.600054 | 3.164421191 | QTL_p_LG5 | surrounding | 4 | 3 |
| Contig4972_3186_all | LG5 | 53.800055 | 3.164816137 | QTL_p_LG5 | surrounding | 3 | 4 |
| Contig31054c_264_04 | LG5 | 54.000056 | 3.166887464 | QTL_p_LG5 | surrounding | 4 | 3 |
| Contig4972_1746_02 | LG5 | 54.500057 | 3.452923509 | QTL_p_LG5 | surrounding | 0 | 1 |
| Contig4797_819_all | LG5 | 54.600058 | 3.453171127 | QTL_p_LG5 | surrounding | 0 | 1 |
| Contig21666c_930_05 | LG5 | 54.600059 | 3.453171127 | QTL_p_LG5 | surrounding | 0 | 1 |
| Contig18126c_943_all | LG5 | 64.40006 | 3.339473856 | QTL_p_LG5 | surrounding | 0 | 1 |
| Contig4435_448_05 | LG5 | 64.400061 | 3.339471772 | QTL_p_LG5 | surrounding | 4 | 3 |
| Contig27658c_151_05 | LG5 | 79.200064 | 3.15241668 | QTL_p_LG5 | surrounding | 4 | 3 |

TABLE 4b

| Marker name | Sequence |
|---|---|
| Contig4023_838_04 | AGGAAGACTAAAAAATCTTCCAAAATGTCAAGCAT[C/T]TTGGTCATTTGTGAGAAAAGCAGTACACGGCTTC |
| Contig4014_1233_all | GTGGTTCAGAAGTGTGACAACTTGAAATCTGAGCC[A/G]TCCAAAATTAGTGGGAACCTCTTGTCTGACAATA |
| Contig23581c_155_all | TGTGCAACATATTCATACAAAGCTTGGTCAACATA[A/G]CCTTTGTAAAATATATCCAAACATTTATGTAATT |
| Contig21666c_2330_all | GAAAAGCAGATTGCCTACCATACTTGTGTTGCATT[A/G]CGCCAGTACTTCAGAGCCCATCTTCTTTTGGTTG |
| Contig4435_509_02 | CGATCACACTGATTGCTAATGAGCCTGCAAACACA[C/T]TTGATGTTATAGAAGCTGCAACCAAAGCTAATCG |
| Contig33097c_465_03 | GATGGCCTGGAAGGAGATGCTATGGTTCTGCAATT[A/G]GAGTCCCAGATTCTTTACCGATTAATGGACATGG |
| Contig48792c_899_all | TCTACAATTCGAGCAATTACTGATCTAATGTCAAA[A/G]GGCTGTCTATGATCAGTTGGCGCAATAGATCGAA |
| Contig981_343_all | ATATCTCGAATCAATTGCTCGTTGGAACCGAGATC[A/G]CCCTCCGCCAAAGCTTCTCTCAACTCTTCAATCT |
| Contig11367_195_all | GTGATGGTGGCTGTATTTAGAATTCTAAAAGACTC[A/G]CTTTCAGAAAAAACCTTCCACCTTATAGAGTTGCT |
| Contig48792c_1070_all | CCAATTTCCAGGCCATGAAGCTCATCATTTGCGAA[A/G]TGATCTGAAACACCAGATATCTTACAATGCAAGG |
| Contig3277c_1388_all | ACTGCATCAAGAAGTTTCTTCCATTCTCGTGCAGC[A/G]TCAAGACTTGCATACACCTCAAGGTCCTTCAGAA |
| Contig11042_1125_all | CACAATGCTAGTAACAAAGTTGGAGCATGCTTCAG[A/C]TATGACTGAATCCACGCAAGGAAGGGGTCCATAT |
| Contig4435_448_05 | GAGCATCTGGTGTCTGCCGTAGCAAATGTAGAGTT[A/G]TCTATTATATGTAGCTTACCTTCATCGATCACAC |
| Contig38099c_803_all | ATGCCAGCGCGACGGATGAGGGCGAGGTTTAGCTC[A/G]GCCTCAGTTAGGAGCATGTCAGCACTAGGATTGC |
| Contig15039c_854_all | CGCCAACCACAAAATAATATCCGTTCTGGAGACTT[G/T]GGACCAATAAAATCCTTGGGCAGATATCCTCCCT |
| Contig11042_2095_12 | TCTCGGATACCATGATCAAATACCTCTTGATAGTG[C/T]GCTTTCCATGCAGTATCATGGGTGGCGTAAAAAGA |
| Contig2023_279_all | ACCTCAATGGAGCTACAACCTGGTTCTTTCTGAAT[A/C]CCTTGCTCCTTCATCATTGCCCGCATCCTGACAG |
| Contig12710c_2266_all | ACCAGTGGTGTAGCTGGAAATCCTAGAATGGCACC[A/C]GATGCAGCTGAAATACAGCTTAAGAGCCCAAACTG |
| Contig2745_618_all | CAGCCTAGTGAAACATAGAAGAGCGTGGTGACCCC[A/G]ATGCCATACAGGGTTGCTCTTTGCATTGTTTTGT |
| Contig12710c_1822_05 | GCTCAGCAATATGGAGGCTCTGCTGCACATAATAC[G/T]GTTTTCCCAGAGAGGCAAGGGAAATGGAAGGCTA |
| Contig10052_518_04 | ACTGGATCGAAGGTCAGTAAATGATCCGGCCCGCG[A/G]CTCAGGCAGTAGACCAATCCATCCAGATATGCCG |
| Contig4882_2237_all | GTGTCGGAGGAGACTGGTAGGAAGAAGTTCTCTCT[A/G]TGGGCGTACGAGGAATATGAAAGTGGAAACATTGC |
| Contig4972_2787_all | CAGTCCGGTAACACAATTACAAGTTCTTTTAATAC[A/G]GAGAAAGCTCCAACCTTGGTTTTGATGGACTTTT |
| Contig4972_3573_all | GCAAGAGATGCAATACAAGAAGCTGACTTCTTTCT[A/G]ACACTGGCTTGATTGACGACCAACTGGGATATAA |
| Contig19967c_129_all | ATCTTATCTTCACTTGCTACTCCTGAACAATCTAT[A/G]GCTATAATGGATCTCATTGAAGCACGTTGGGAAG |
| Contig283sc_148_04 | CATTTCACAAGGAACTCAAGATCCCCTTTGTTCAA[C/T]GCAGAATTGTGAATATCCATGATTTCGCATGGATG |
| Contig21666c_4757_all | CACACATGTCACCAGACTCCAGTTACAATTGTTCA[A/G]TCAGCTTTATCTGGTGAAATCCAGATGCTTCTTT |
| Contig9996_428_02 | TTGCACTCTCGTGGTTTCGTCTGCGGATGCTTGAC[A/G]CCAGCTTGCTTTCGTCTTGATGAGTATGGGCATT |
| Contig9550_487_03 | TAAGAACAATTCCGATGATCAGAAGAGAGTTGCAC[A/G]GAATGACTCCGATGGAACAGACAGTAACAAAGAG |
| Contig21666c_4350_all | CCGAACTACAGTCTGGAGCTATGGAGCGGACAACT[C/T]TGGACTCTTTGGTTGTGCAGTACTTGAAGCACCA |
| Contig23932c_1258_04 | CAGGGTAAAATATCTGAAGAATTAATAGATCGCCT[C/T]ATGGGGACTCTTGGTCATTTGGTGCAGCGCAATAC |
| Contig4797_1761_05 | CGAAAACCCTACCCTTATCTCCTCACACCGCAAGA[G/T]GCTGCCTTTCCATCCAAAACCTGTGATTTCTGGA |
| Contig4794_1130_03 | TCAACAAATTCGGGCTTGATGTCATACAGGAAGCG[C/T]GATATCCTATGCCAAACTCCTTTCGGTGCTAGAG |
| Contig12710c_1627_all | TTCCAAAGTGGAGTCCTTCGTACTAACTGTATCGA[C/T]TGCTTGGATCGTACGAACGTTGCACAGTATGCAT |
| Contig12710c_2551_02 | GCACTAATTTATGACCAAGACGAAAACAGTGAGGT[G/T]TACAAGCATTATTCTGAACTATGTCAGGGACCTG |
| Contig14450sc_358_02 | AGCCATCTCAAAAAGCTGAACGCACACTATTTGTT[A/C]ATCGAATCCCTCAAAACGTCAATAGGCCACGGGCC |
| Contig1477sc_165_03 | ATTTCATGTGTGAAAATTCATCTCCATCCGAATAT[A/G]TAGCTTCAAATGGACTTAACAACAGACCTGATGAG |
| Contig10987_638_03 | AATGTTGGGGCATGTGGTGAAGCTGAAAAGTTGAT[C/T]GAAAATAGCAACCCTGTGGAGATTCATTTCTCAC |
| Contig11321c_252_03 | GGAGTAGAATATCAGGCTGAACGTATTTTGCTTGA[G/T]CTACAGACGAATGACAAATATTGTGATGAGGACT |
| Contig10685_81_05 | ATAAATCAAAAGAGCGACAATATCCATTCTTATCA[C/T]GAGATTATTAAGGTTGCAAGACATTTTTAAAATCT |
| Contig5429_1938_all | GTGTTGCACCCGAGACGTTATCTGAAGATGGCATT[A/G]CTAGAGTTTCTAAGACAGAGAGCAAAAGCGACAC |
| Contig9806sc_215_12 | CATCAACAGAAACACTGCCATGCTCAATGGTGTAG[A/C]ATTGTGGTTCATTGAATCTCCAGAAAGAAATATT |
| Contig1858_2184_all | TTTTATTTTCTTAAAATGCCGTTCTCTCCTGTAAT[G/T]GTTTTGGTTTCTCGAACCGAGATCACGATCTCCA |
| Contig1858_1186_all | TGACAACCGCTGAACATGAACTTCCTATGAGATTT[A/C]TGCTTTGAACCTTTATTGGGCACTTCTAACCCTC |
| Contig5429_1938_all | GTGTTGCACCCGAGACGTTATCTGAAGATGGCATT[A/G]CTAGAGTTTCTAAGACAGAGAGCAAAAGCGACAC |
| Contig47217c_830_all | GCGTTGGGGATATTCTTTAGAGCATGAAAGCAATG[C/T]CAGTTAGGTTTGCTTAGGAGTTTAATGATCTGTT |
| Contig10987_572_all | AGGCGTAAAGGGGTCACCGGTAAGAAAGTAGCAAA[G/T]ACTACAGCTCCAAAGTCCGCAGAATGTAGTAATA |
| Contig1858_1910_all | TCCAATCCTTCCCCTCTTTCCACATCATGGCATGA[C/T]TGTATCGGTTTGTAATGCCAGGCTGCAGAACTTG |
| Contig9145_477_03 | TATACGAGTAGCCATGTTGATAACCCACTATGCCG[C/T]GAAAGCTTAGATATGGAACTATCAACTGCTGCAG |
| Contig25540c_69_06 | TGGTATACAAGTATGTCAGCGTGTGTCCACTGAAA[A/G]CAGAAAGAAACTGAAAGCGATCAGATATTATAAT |
| Contig631_1175_03 | ATACCTTTATATACCTTCCCATATCCTCCTTGTCC[A/C]ACTTGAGCAGAGTTGCTGAAATTATTTGTAGCTA |
| Contig11637c_525_all | CTTGATCCAGAAAGGTCGCTTGTACTGGGACCTTG[G/T]CCTACTAGAGTTTGTTGAGAAGAAGCTGTTTTCC |
| Contig38500c_86_03 | GAATATTGTGTACCTCTCGTTCAAAGGGAAGTACA[A/G]GTTATAAAGCAAAATAACATCATGATCAGGCCTG |
| Contig669_218_04 | TCTGCTTCGGCGATTCATCGTCCAAATCATCGGAG[A/G]GAGGTGAGCTCATCAACTTCCCAGCAACAGTGTG |
| Contig3891_979_04 | ATACTATCCCCAGGCTCTAATGTTATATGCTTAGC[A/G]TCCAATGATGAGAGAGCTTTGGTCCATGGCCTTC |
| Contig5523_3142_03 | ATTTGATGAGGATCTTGTTTCATGCGTTTTGAAGC[A/G]GTTATTACTTTCAAATGGCCAAGGGCTGATGCAA |
| Contig5523_2455_04 | CCTTCTAATCTGTTCCTCAATATTCTTCCTCTATC[A/G]CAGACAAAATAAAATAAACAACTCAGAGCAGATG |
| Contig11449_1176_all | CTGCGTCCAGTTTGGTTGAAGTAATCTTTGCAGTC[A/G]CTCATAATCGCATCCAAAGGATAGGCCTTTGCAC |
| Contig5523_3453_03 | AAGGAGATCAAAGAAAATATATTTACTATCATCGT[A/G]TACATCATCAGTTCCAATATTAGATGCATCCAGT |
| Contig47887c_978_06 | TGATTTGATGGCTGCGTTGACAAATTTAAACCTGA[G/T]CCATTGAGCAATTTCCTCGGATGGGAAAGCAAGG |
| Contig11054c_440_all | ATGTCATCAGATTCATATCTCCAGTTCTGCTCATC[A/C]TTCCGATAACTAGGCATGGATGAACTTGATCTCC |
| Contig29740c_640_04 | AACCTTATGGCCACACGCAATCTCAACACTTTGAG[C/T]ATGCCGTTAGTGAGCCAACCTGTTACATGTGAAC |
| Contig12758c_410_04 | GAGAGACCTGATTGGCTGTTGTTCTCATGGATCAA[C/T]AAAGATGAAGATGATGATCTGGCCCTTCCAAATA |
| Contig8038_998_all | TTGTCAAGGTAGTCGGCCCTGTAAATCTTTCCAGT[C/T]GTAGCTTCATTAGCAATGTTTTCCCAGCGAATTG |
| Contig9761_813_all | TTTGGAAAGCTGCTTTAAGTTCCACAAGAGCATCA[C/T]AATTTATTTCTTCTAGGTAATGAAAATGTGGAAC |
| Contig2582c_1172_all | GCTGCTGCCCTAGGAAGGGTCCTAATGCTGGACCT[C/T]GTAATTAGGAATGAAGATAGACTTCCTTGTCGCC |
| Contig19739c_664_02 | TGATTAGTTTATTGGATGAGGTTATGGGATAGTAA[A/C]ATTTCTATTTTGTGTAGCTGATTTGTGGTCCTGGT |
| Contig1813sc_151_07 | AAGTGGTCGCTAGAGTTCGTGAGGGTCTTGGTCAT[A/G]CCACCAACAACCAGGCTGAATATGAGGGCTTGAA |
| Contig26701c_392_07 | CACAAGGCCTCTCTTCCTATATAGCGTTTGATGAG[A/C]AGCTCATTCCATTTAAGCTCTTCCTTCCATACAA |
| Contig11429_3551_07 | GCAAAGCTACTATATCAAGAGCTTTTCAGCGTAGG[A/C]ATATGTCTCAAGTTTGAAACAGAGGTTCGTTAAA |
| Contig2648_3441_all | GATTTAAGTGTATCAAGTAGCCATGACACAAGATC[C/T]GGAAAGTGTTCCTCGCCCATTCCTTTGATAAGAG |
| Contig50234c_876_05 | AGCTGCTACAAGTAATAATGGCAACCATTGGTTTC[A/G]TATTCGTGTACCTCTGCATCACTAAAGGCGAAGA |
| Contig9319_471_23 | TTCTCGGAGTAGACAACATTCGTTGCAGGGTCGAC[A/C]GTGGACTTAATGGCGTCGAGAATTGTGGTTCCAG |
| Contig37704c_728_07 | CGTCGGACAGCTTGTAACTGTACAGTAATTTATCT[C/T]TTCTCTACGTTTCTCTTGAGATCATGGATTGAAC |
| Contig27369sc_156_02 | TTCCTTTGCCGAAAGCTGTGCCCTCCGATTAAAGG[A/G]ATGGATGTCAGCCCTCTTATTGCATTTTGGGTCAT |
| Contig15834c_1178_all | TCTCAGGCGGTCCGCGTCAGCCCGTTCCAAGGAAT[C/T]GAGCCCAAGTGCAGTAGTTCACGCTTCTACGTTG |
| Contig1858_2184_all | TTTTATTTTCTTAAAATGCCGTTCTCTCCTGTAAT[G/T]GTTTTGGTTTCTCGAACCGAGATCACGATCTCCA |
| Contig10987_572_all | AGGCGTAAAGGGGTCACCGGTAAGAAAGTAGCAAA[G/T]ACTACAGCTCCAAAGTCCGCAGAATGTAGTAATA |
| Contig32205c_428_02 | ATGGTAAAATTTTCAGAGAACTTTTTTACAGCTGC[A/G]TCTATGTTTTTCTTCTGATTATATCCAACGGTAAA |
| Contig3804_255_04 | CACAACCCATTGATCAACACACAAAACGTGTCAAC[A/G]CTCGGAACAAAATGTTTCAACTTCATGTACTTCC |
| Contig9821_1567_all | CACAGATTGTCAGACAGGTTATATCAAAGTTCCCC[A/G]GGATAAGGTTAATATACAAACGACACTAATGATG |
| Contig21666c_1131_07 | GGACCAGAGGGAAAGGAAAGACAACTGAAAGTACC[A/G]CAGAAAATGAAAGGGCTGTAGTCTCTCCAAGTTC |
| Contig4972_3186_all | TCCTCTTCCTCTTGGCCTTCATTGTCAGTATCCTC[A/G]TCCATGTTGTCAGTGAAATTCGGGTCATAGCTTA |
| Contig31054c_264_04 | CTTGCCAACAAGACTAGAGACTTCTCTGGAACCTG[C/T]ACAAAGACACGTCAATATGAACTTCAAGCTATAC |
| Contig4972_1746_02 | TTGTTATCTCCAGCTGCCAAGCAAAGTACAGCAAC[A/G]CATTGTGCTATTGAACAAAGAGCCTGCTTAGCCAA |
| Contig4797_819_all | CTCTCGGAGGCAGGAGGATTGCCTGTGGTTGTCCT[C/T]GCATCTCGCTTGCAGACGGGGCAAAATGTTCTCCA |
| Contig21666c_930_05 | CTGATCCTTCAGCAGAAGTCGTGGGTATGTATGAG[A/G]TGGTCGGGGAGGGTGCTGATCTCACTGATGGATG |
| Contig18126c_943_all | GATGCTCTTCACACCATGCATGAAGGGAAATTCCT[G/T]CATCTCCCTGTTGTAGACAGAGATGGGAACATTGT |
| Contig4435_448_05 | GAGCATCTGGTGTCTGCCGTAGCAAATGTAGAGTT[A/G]TCTATTATATGTAGCTTACCTTCATCGATCACAC |
| Contig27658c_151_05 | ATGCTCTACGCTCAGATTGATTCAGTTTACGCGGC[A/G]ATGAAAGCAATGGGGCATACAGACATTGGTGTCA |

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An Alstroemeria L. plant, comprising a single flower per stem, which flower comprises more than 6 tepals.

2. The Alstroemeria L. plant as claimed in claim 1, wherein the flower comprises 10 to 40 tepals.

3. The Alstroemeria L. plant as claimed in claim 1, wherein the flower comprises 15 to 35 tepals.

4. The Alstroemeria L. plant as claimed in claim 1, wherein the flower comprises 20 to 30 tepals.

5. The Alstroemeria L. plant as claimed in claim 1, wherein the plant comprises no peduncles.

6. The Alstroemeria L. plant as claimed in claim 1, wherein pedicels are absent.

7. The Alstroemeria L. plant as claimed in claim 1, wherein the flower is not zygomorphic.

8. The Alstroemeria L. plant as claimed in claim 1, wherein the flower is actinomorphic.

9. The Alstroemeria L. plant as claimed in claim 1, wherein the flower comprises a rudimentary or malformed ovary.

10. The Alstroemeria L. plant as claimed in claim 1, wherein the tepals are arranged in a dense spiral structure.

11. The Alstroemeria L. plant as claimed in claim 1, wherein a part of a leaf shows the flower colour.

12. The Alstroemeria L. plant as claimed in claim 11, wherein the leaves are arranged in an elongated spiral structure.

13. The Alstroemeria L. plant as claimed in claim 1, wherein a stamen is an integral part of a tepal.

14. The Alstroemeria L. plant as claimed in claim 1, wherein the pistils are degenerated.

15. The Alstroemeria L. plant as claimed in claim 1, wherein a stem comprises 1 to 3 additional flowers, which are each located on a pedicel.

16. The Alstroemeria L. plant as claimed in claim 1, wherein the plant produces a single flower per stem during the complete flowering period.

17. The Alstroemeria L. plant as claimed in claim 1, comprising a single flower per stem, which flower comprises more than six tepals, lacks peduncles and at least partially lacks pedicels and which plant produces a single flower per stem during the complete flowering period.

18. A QTL contributing to the double flower trait, selected from QTL_p_LG5, QTL_p_LG10, QTL_p_LG22_1, QTL_p_LG22_2, QTL_m_LG3.

19. A molecular marker for detecting the presence of a QTL of claim 18.

20. The molecular marker of claim 19 for detecting the presence of QTL_p_LG5, comprising the sequence
TCTCAGGCGGTCCGCGTCAGCCCGTTCCAAGGAAT[C/T]GAGCCCAAGT
GCAGTAGTTCACGCTTCTACGTTG.

21. The molecular marker of claim 19 for detecting the presence of QTL_p_LG10, comprising the sequence
CTTGATCCAGAAAGGTCGCTTGTACTGGGACCTTG[G/T]CCTACTAGAG
TTTGTTGAGAAGAAGCTGTTTTCC.

22. The molecular marker of claim 19 for detecting the presence of QTL_p_LG22_1, comprising the sequence
GCAAAGCTACTATATCAAGAGCTTTTCAGCGTAGG[A/C]ATATGTCTCA
AGTTTGAAACAGAGGTTCGTTAAA.

23. The molecular marker of claim 19 for detecting the presence of QTL_p_LG22_2, comprising the sequence
TTCCTTTGCCGAAAGCTGTGCCCTCCGATTAAAGG[A/G]ATGGATGTCA
GCCCTCTTATTGCATTTTGGGTCAT.

24. The molecular marker of claim 19 for detecting the presence of QTL_m_LG3, comprising the sequence
TATACGAGTAGCCATGTTGATAACCCACTATGCCG[C/T]GAAAGCTTAG
ATATGGAACTATCAACTGCTGCAG.


SEQUENCE LISTING (180 sequences)

SEQ ID NO: 1 - DNA, 70 bp, Alstroemeria sp.
  source 1..70: mol_type = genomic DNA
aggaagactaaaaaatcttccaaaatgtcaagcatcttggtcatttgtgagaaaagcagtacacggcttc

SEQ ID NO: 2 - DNA, 70 bp, Alstroemeria sp.
  source 1..70: mol_type = genomic DNA
gtggttcagaagtgtgacaacttgaaatctgagccatccaaaattagtgggaacctcttgtctgacaata

SEQ ID NO: 3 - DNA, 70 bp, Alstroemeria sp.
  source 1..70: mol_type = genomic DNA
tgtgcaacatattcatacaaagcttggtcaacataacctttgtaaaatatatccaaacatttatgtaatt

SEQ ID NO: 4 - DNA, 70 bp, Alstroemeria sp.
  source 1..70: mol_type = genomic DNA
gaaaagcagattgcctaccatacttgtgttgcattacgccagtacttcagagcccatcttcttttggttg

SEQ ID NO: 5 - DNA, 70 bp, Alstroemeria sp.
  source 1..70: mol_type = genomic DNA
cgatcacactgattgctaatgagcctgcaaacacacttgatgttatagaagctgcaaccaaagctaatcg

SEQ ID NO: 6 - DNA, 70 bp, Alstroemeria sp.
  source 1..70: mol_type = genomic DNA
gatggcctggaaggagatgctatggttctgcaattagagtcccagattctttaccgattaatggacatgg

SEQ ID NO: 7 - DNA, 70 bp, Alstroemeria sp.
  source 1..70: mol_type = genomic DNA
tctacaattcgagcaattactgatctaatgtcaaaaggctgtctatgatcagttggcgcaatagatcgaa

SEQ ID NO: 8 - DNA, 70 bp, Alstroemeria sp.
  source 1..70: mol_type = genomic DNA
atatctcgaatcaattgctcgttggaaccgagatcaccctccgccaaagcttctctcaactcttcaatct

SEQ ID NO: 9 - DNA, 71 bp, Alstroemeria sp.
  source 1..71: mol_type = genomic DNA
gtgatggtggctgtatttagaattctaaaagactcactttcagaaaaaaccttccaccttatagagttgct

SEQ ID NO: 10 - DNA, 70 bp, Alstroemeria sp.
  source 1..70: mol_type = genomic DNA
ccaatttccaggccatgaagctcatcatttgcgaaatgatctgaaacaccagatatcttacaatgcaagg

SEQ ID NO: 11 - DNA, 70 bp, Alstroemeria sp.
  source 1..70: mol_type = genomic DNA
actgcatcaagaagtttcttccattctcgtgcagcatcaagacttgcatacacctcaaggtccttcagaa

SEQ ID NO: 12 - DNA, 70 bp, Alstroemeria sp.
  source 1..70: mol_type = genomic DNA
cacaatgctagtaacaaagttggagcatgcttcagatatgactgaatccacgcaaggaaggggtccatat

SEQ ID NO: 13 - DNA, 70 bp, Alstroemeria sp.
  source 1..70: mol_type = genomic DNA
gagcatctggtgtctgccgtagcaaatgtagagttatctattatatgtagcttaccttcatcgatcacac

SEQ ID NO: 14 - DNA, 70 bp, Alstroemeria sp.
  source 1..70: mol_type = genomic DNA
atgccagcgcgacggatgagggcgaggtttagctcagcctcagttaggagcatgtcagcactaggattgc

SEQ ID NO: 15 - DNA, 70 bp, Alstroemeria sp.
  source 1..70: mol_type = genomic DNA
cgccaaccacaaaataatatccgttctggagacttgggaccaataaaatccttgggcagatatcctccct

SEQ ID NO: 16 - DNA, 71 bp, Alstroemeria sp.
  source 1..71: mol_type = genomic DNA
tctcggataccatgatcaaatacctcttgatagtgcgctttccatgcagtatcatgggtggcgtaaaaaga

SEQ ID NO: 17 - DNA, 70 bp, Alstroemeria sp.
  source 1..70: mol_type = genomic DNA
acctcaatggagctacaacctggttctttctgaataccttgctccttcatcattgcccgcatcctgacag

SEQ ID NO: 18 - DNA, 71 bp, Alstroemeria sp.
  source 1..71: mol_type = genomic DNA
accagtggtgtagctggaaatcctagaatggcaccagatgcagctgaaatacagcttaagagcccaaactg

SEQ ID NO: 19 - DNA, 70 bp, Alstroemeria sp.
  source 1..70: mol_type = genomic DNA
cagcctagtgaaacatagaagagcgtggtgaccccaatgccatacagggttgctctttgcattgttttgt

SEQ ID NO: 20 - DNA, 70 bp, Alstroemeria sp.
  source 1..70: mol_type = genomic DNA
gctcagcaatatggaggctctgctgcacataatacggttttcccagagaggcaagggaaatggaaggcta

SEQ ID NO: 21 - DNA, 70 bp, Alstroemeria sp.
  source 1..70: mol_type = genomic DNA
actggatcgaaggtcagtaaatgatccggcccgcgactcaggcagtagaccaatccatccagatatgccg

SEQ ID NO: 22 - DNA, 71 bp, Alstroemeria sp.
  source 1..71: mol_type = genomic DNA
gtgtcggaggagactggtaggaagaagttctctctatgggcgtacgaggaatatgaaagtggaaacattgc

SEQ ID NO: 23 - DNA, 70 bp, Alstroemeria sp.
  source 1..70: mol_type = genomic DNA
cagtccggtaacacaattacaagttcttttaatacagagaaagctccaaccttggttttgatggactttt

SEQ ID NO: 24 - DNA, 70 bp, Alstroemeria sp.
  source 1..70: mol_type = genomic DNA
gcaagagatgcaatacaagaagctgacttctttctaacactggcttgattgacgaccaactgggatataa

SEQ ID NO: 25 - DNA, 70 bp, Alstroemeria sp.
  source 1..70: mol_type = genomic DNA
atcttatcttcacttgctactcctgaacaatctatagctataatggatctcattgaagcacgttgggaag

SEQ ID NO: 26 - DNA, 71 bp, Alstroemeria sp.
  source 1..71: mol_type = genomic DNA
catttcacaaggaactcaagatcccctttgttcaacgcagaattgtgaatatccatgatttcgcatggatg

SEQ ID NO: 27 - DNA, 70 bp, Alstroemeria sp.
  source 1..70: mol_type = genomic DNA
cacacatgtcaccagactccagttacaattgttcaatcagctttatctggtgaaatccagatgcttcttt

SEQ ID NO: 28 - DNA, 70 bp, Alstroemeria sp.
  source 1..70: mol_type = genomic DNA
ttgcactctcgtggtttcgtctgcggatgcttgacaccagcttgctttcgtcttgatgagtatgggcatt

SEQ ID NO: 29 - DNA, 70 bp, Alstroemeria sp.
  source 1..70: mol_type = genomic DNA
taagaacaattccgatgatcagaagagagttgcacagaatgactccgatggaacagacagtaacaaagag

SEQ ID NO: 30 - DNA, 70 bp, Alstroemeria sp.
  source 1..70: mol_type = genomic DNA
ccgaactacagtctggagctatggagcggacaactctggactctttggttgtgcagtacttgaagcacca

SEQ ID NO: 31 - DNA, 71 bp, Alstroemeria sp.
  source 1..71: mol_type = genomic DNA
cagggtaaaatatctgaagaattaatagatcgcctcatggggactcttggtcatttggtgcagcgcaatac

SEQ ID NO: 32 - DNA, 70 bp, Alstroemeria sp.
  source 1..70: mol_type = genomic DNA
cgaaaaccctacccttatctcctcacaccgcaagaggctgcctttccatccaaaacctgtgatttctgga

SEQ ID NO: 33 - DNA, 70 bp, Alstroemeria sp.
  source 1..70: mol_type = genomic DNA
tcaacaaattcgggcttgatgtcatacaggaagcgcgatatcctatgccaaactcctttcggtgctagag

SEQ ID NO: 34 - DNA, 70 bp, Alstroemeria sp.
  source 1..70: mol_type = genomic DNA
ttccaaagtggagtccttcgtactaactgtatcgactgcttggatcgtacgaacgttgcacagtatgcat

SEQ ID NO: 35 - DNA, 70 bp, Alstroemeria sp.
  source 1..70: mol_type = genomic DNA
gcactaatttatgaccaagacgaaaacagtgaggtgtacaagcattattctgaactatgtcagggacctg

SEQ ID NO: 36 - DNA, 71 bp, Alstroemeria sp.
  source 1..71: mol_type = genomic DNA
agccatctcaaaaagctgaacgcacactatttgttaatcgaatccctcaaaacgtcaataggccacgggcc

SEQ ID NO: 37 - DNA, 71 bp, Alstroemeria sp.
  source 1..71: mol_type = genomic DNA
atttcatgtgtgaaaattcatctccatccgaatatatagcttcaaatggacttaacaacagacctgatgag

SEQ ID NO: 38 - DNA, 70 bp, Alstroemeria sp.
  source 1..70: mol_type = genomic DNA
aatgttggggcatgtggtgaagctgaaaagttgatcgaaaatagcaaccctgtggagattcatttctcac

SEQ ID NO: 39 - DNA, 70 bp, Alstroemeria sp.
  source 1..70: mol_type = genomic DNA
ggagtagaatatcaggctgaacgtattttgcttgagctacagacgaatgacaaatattgtgatgaggact

SEQ ID NO: 40 - DNA, 71 bp, Alstroemeria sp.
  source 1..71: mol_type = genomic DNA
ataaatcaaaagagcgacaatatccattcttatcacgagattattaaggttgcaagacatttttaaaatct

SEQ ID NO: 41 - DNA, 70 bp, Alstroemeria sp.
  source 1..70: mol_type = genomic DNA
gtgttgcacccgagacgttatctgaagatggcattactagagtttctaagacagagagcaaaagcgacac

SEQ ID NO: 42 - DNA, 70 bp, Alstroemeria sp.
  source 1..70: mol_type = genomic DNA
catcaacagaaacactgccatgctcaatggtgtagaattgtggttcattgaatctccagaaagaaatatt

SEQ ID NO: 43 - DNA, 70 bp, Alstroemeria sp.
  source 1..70: mol_type = genomic DNA
ttttattttcttaaaatgccgttctctcctgtaatggttttggtttctcgaaccgagatcacgatctcca

SEQ ID NO: 44 - DNA, 70 bp, Alstroemeria sp.
  source 1..70: mol_type = genomic DNA
tgacaaccgctgaacatgaacttcctatgagatttatgctttgaacctttattgggcacttctaaccctc

SEQ ID NO: 45 - DNA, 70 bp, Alstroemeria sp.; identical to SEQ ID NO: 41
  source 1..70: mol_type = genomic DNA

SEQ ID NO: 46 - DNA, 70 bp, Alstroemeria sp.
  source 1..70: mol_type = genomic DNA
gcgttggggatattctttagagcatgaaagcaatgccagttaggtttgcttaggagtttaatgatctgtt

SEQ ID NO: 47 - DNA, 70 bp, Alstroemeria sp.
  source 1..70: mol_type = genomic DNA
aggcgtaaaggggtcaccggtaagaaagtagcaaagactacagctccaaagtccgcagaatgtagtaata

SEQ ID NO: 48 - DNA, 70 bp, Alstroemeria sp.
  source 1..70: mol_type = genomic DNA
tccaatccttcccctctttccacatcatggcatgactgtatcggtttgtaatgccaggctgcagaacttg

SEQ ID NO: 49 - DNA, 70 bp, Alstroemeria sp.
  source 1..70: mol_type = genomic DNA
tatacgagtagccatgttgataacccactatgccgcgaaagcttagatatggaactatcaactgctgcag

SEQ ID NO: 50 - DNA, 70 bp, Alstroemeria sp.
  source 1..70: mol_type = genomic DNA
tggtatacaagtatgtcagcgtgtgtccactgaaaacagaaagaaactgaaagcgatcagatattataat

SEQ ID NO: 51 - DNA, 70 bp, Alstroemeria sp.
  source 1..70: mol_type = genomic DNA
atacctttatataccttcccatatcctccttgtccaacttgagcagagttgctgaaattatttgtagcta

SEQ ID NO: 52 - DNA, 70 bp, Alstroemeria sp.
  source 1..70: mol_type = genomic DNA
cttgatccagaaaggtcgcttgtactgggaccttggcctactagagtttgttgagaagaagctgttttcc

SEQ ID NO: 53 - DNA, 70 bp, Alstroemeria sp.
  source 1..70: mol_type = genomic DNA
gaatattgtgtacctctcgttcaaagggaagtacaagttataaagcaaaataacatcatgatcaggcctg

SEQ ID NO: 54 - DNA, 70 bp, Alstroemeria sp.
  source 1..70: mol_type = genomic DNA
tctgcttcggcgattcatcgtccaaatcatcggagagaggtgagctcatcaacttcccagcaacagtgtg

SEQ ID NO: 55 - DNA, 70 bp, Alstroemeria sp.
  source 1..70: mol_type = genomic DNA
atactatccccaggctctaatgttatatgcttagcatccaatgatgagagagctttggtccatggccttc

SEQ ID NO: 56 - DNA, 70 bp, Alstroemeria sp.
  source 1..70: mol_type = genomic DNA
atttgatgaggatcttgtttcatgcgttttgaagcagttattactttcaaatggccaagggctgatgcaa

SEQ ID NO: 57 - DNA, 70 bp, Alstroemeria sp.
  source 1..70: mol_type = genomic DNA
ccttctaatctgttcctcaatattcttcctctatcacagacaaaataaaataaacaactcagagcagatg

SEQ ID NO: 58 - DNA, 70 bp, Alstroemeria sp.
  source 1..70: mol_type = genomic DNA
ctgcgtccagtttggttgaagtaatctttgcagtcactcataatcgcatccaaaggataggcctttgcac

SEQ ID NO: 59 - DNA, 70 bp, Alstroemeria sp.
  source 1..70: mol_type = genomic DNA
aaggagatcaaagaaaatatatttactatcatcgtatacatcatcagttccaatattagatgcatccagt

SEQ ID NO: 60 - DNA, 70 bp, Alstroemeria sp.
  source 1..70: mol_type = genomic DNA
tgatttgatggctgcgttgacaaatttaaacctgagccattgagcaatttcctcggatgggaaagcaagg

SEQ ID NO: 61 - DNA, 70 bp, Alstroemeria sp.
  source 1..70: mol_type = genomic DNA
atgtcatcagattcatatctccagttctgctcatcattccgataactaggcatggatgaacttgatctcc

SEQ ID NO: 62 - DNA, 70 bp, Alstroemeria sp.
  source 1..70: mol_type = genomic DNA
aaccttatggccacacgcaatctcaacactttgagcatgccgttagtgagccaacctgttacatgtgaac

SEQ ID NO: 63 - DNA, 70 bp, Alstroemeria sp.
  source 1..70: mol_type = genomic DNA
gagagacctgattggctgttgttctcatggatcaacaaagatgaagatgatgatctggcccttccaaata

SEQ ID NO: 64 - DNA, 70 bp, Alstroemeria sp.
  source 1..70: mol_type = genomic DNA
ttgtcaaggtagtcggccctgtaaatctttccagtcgtagcttcattagcaatgttttcccagcgaattg

SEQ ID NO: 65 - DNA, 70 bp, Alstroemeria sp.
  source 1..70: mol_type = genomic DNA
tttggaaagctgctttaagttccacaagagcatcacaatttatttcttctaggtaatgaaaatgtggaac

SEQ ID NO: 66 - DNA, 70 bp, Alstroemeria sp.
  source 1..70: mol_type = genomic DNA
gctgctgccctaggaagggtcctaatgctggacctcgtaattaggaatgaagatagacttccttgtcgcc

SEQ ID NO: 67 - DNA, 71 bp, Alstroemeria sp.
  source 1..71: mol_type = genomic DNA
tgattagtttattggatgaggttatgggatagtaaaatttctattttgtgtagctgatttgtggtcctggt

SEQ ID NO: 68 - DNA, 70 bp, Alstroemeria sp.
  source 1..70: mol_type = genomic DNA
aagtggtcgctagagttcgtgagggtcttggtcataccaccaacaaccaggctgaatatgagggcttgaa

SEQ ID NO: 69 - DNA, 70 bp, Alstroemeria sp.
  source 1..70: mol_type = genomic DNA
cacaaggcctctcttcctatatagcgtttgatgagaagctcattccatttaagctcttccttccatacaa

SEQ ID NO: 70 - DNA, 70 bp, Alstroemeria sp.
  source 1..70: mol_type = genomic DNA
gcaaagctactatatcaagagcttttcagcgtaggaatatgtctcaagtttgaaacagaggttcgttaaa

SEQ ID NO: 71 - DNA, 70 bp, Alstroemeria sp.
  source 1..70: mol_type = genomic DNA
gatttaagtgtatcaagtagccatgacacaagatccggaaagtgttcctcgcccattcctttgataagag

SEQ ID NO: 72 - DNA, 70 bp, Alstroemeria sp.
  source 1..70: mol_type = genomic DNA
agctgctacaagtaataatggcaaccattggtttcatattcgtgtacctctgcatcactaaaggcgaaga

SEQ ID NO: 73 - DNA, 70 bp, Alstroemeria sp.
  source 1..70: mol_type = genomic DNA
ttctcggagtagacaacattcgttgcagggtcgacagtggacttaatggcgtcgagaattgtggttccag

SEQ ID NO: 74 - DNA, 70 bp, Alstroemeria sp.
  source 1..70: mol_type = genomic DNA
cgtcggacagcttgtaactgtacagtaatttatctcttctctacgtttctcttgagatcatggattgaac

SEQ ID NO: 75 - DNA, 71 bp, Alstroemeria sp.
  source 1..71: mol_type = genomic DNA
ttcctttgccgaaagctgtgccctccgattaaaggaatggatgtcagccctcttattgcattttgggtcat

SEQ ID NO: 76 - DNA, 70 bp, Alstroemeria sp.
  source 1..70: mol_type = genomic DNA
tctcaggcggtccgcgtcagcccgttccaaggaatcgagcccaagtgcagtagttcacgcttctacgttg

SEQ ID NO: 77 - DNA, 70 bp, Alstroemeria sp.; identical to SEQ ID NO: 43
  source 1..70: mol_type = genomic DNA

SEQ ID NO: 78 - DNA, 70 bp, Alstroemeria sp.; identical to SEQ ID NO: 47
  source 1..70: mol_type = genomic DNA

SEQ ID NO: 79 - DNA, 71 bp, Alstroemeria sp.
  source 1..71: mol_type = genomic DNA
atggtaaaattttcagagaacttttttacagctgcatctatgtttttcttctgattatatccaacggtaaa

SEQ ID NO: 80 - DNA, 70 bp, Alstroemeria sp.
  source 1..70: mol_type = genomic DNA
cacaacccattgatcaacacacaaaacgtgtcaacactcggaacaaaatgtttcaacttcatgtacttcc

SEQ ID NO: 81 - DNA, 70 bp, Alstroemeria sp.
  source 1..70: mol_type = genomic DNA
cacagattgtcagacaggttatatcaaagttccccaggataaggttaatatacaaacgacactaatgatg

SEQ ID NO: 82 - DNA, 70 bp, Alstroemeria sp.
  source 1..70: mol_type = genomic DNA
ggaccagagggaaaggaaagacaactgaaagtaccacagaaaatgaaagggctgtagtctctccaagttc

SEQ ID NO: 83 - DNA, 70 bp, Alstroemeria sp.
  source 1..70: mol_type = genomic DNA
tcctcttcctcttggccttcattgtcagtatcctcatccatgttgtcagtgaaattcgggtcatagctta

SEQ ID NO: 84 - DNA, 70 bp, Alstroemeria sp.
  source 1..70: mol_type = genomic DNA
cttgccaacaagactagagacttctctggaacctgcacaaagacacgtcaatatgaacttcaagctatac

SEQ ID NO: 85 - DNA, 71 bp, Alstroemeria sp.
  source 1..71: mol_type = genomic DNA
ttgttatctccagctgccaagcaaagtacagcaacacattgtgctattgaacaaagagcctgcttagccaa

SEQ ID NO: 86 - DNA, 71 bp, Alstroemeria sp.
  source 1..71: mol_type = genomic DNA
ctctcggaggcaggaggattgcctgtggttgtcctcgcatctcgcttgcagacggggcaaaatgttctcca

SEQ ID NO: 87 - DNA, 70 bp, Alstroemeria sp.
  source 1..70: mol_type = genomic DNA
ctgatccttcagcagaagtcgtgggtatgtatgagatggtcggggagggtgctgatctcactgatggatg

SEQ ID NO: 88 - DNA, 71 bp, Alstroemeria sp.
  source 1..71: mol_type = genomic DNA
gatgctcttcacaccatgcatgaagggaaattcctgcatctccctgttgtagacagagatgggaacattgt

SEQ ID NO: 89 - DNA, 70 bp, Alstroemeria sp.; identical to SEQ ID NO: 13
  source 1..70: mol_type = genomic DNA

SEQ ID NO: 90 - DNA, 70 bp, Alstroemeria sp.
  source 1..70: mol_type = genomic DNA
atgctctacgctcagattgattcagtttacgcggcaatgaaagcaatggggcatacagacattggtgtca

SEQ ID NO: 91 - DNA, 70 bp, Alstroemeria sp.
  source 1..70: mol_type = genomic DNA
aggaagactaaaaaatcttccaaaatgtcaagcattttggtcatttgtgagaaaagcagtacacggcttc

SEQ ID NO: 92 - DNA, 70 bp, Alstroemeria sp.
  source 1..70: mol_type = genomic DNA
gtggttcagaagtgtgacaacttgaaatctgagccgtccaaaattagtgggaacctcttgtctgacaata

SEQ ID NO: 93 - DNA, 70 bp, Alstroemeria sp.
  source 1..70: mol_type = genomic DNA
tgtgcaacatattcatacaaagcttggtcaacatagcctttgtaaaatatatccaaacatttatgtaatt

SEQ ID NO: 94 - DNA, 70 bp, Alstroemeria sp.
  source 1..70: mol_type = genomic DNA
gaaaagcagattgcctaccatacttgtgttgcattgcgccagtacttcagagcccatcttcttttggttg

SEQ ID NO: 95 - DNA, 70 bp, Alstroemeria sp.
  source 1..70: mol_type = genomic DNA
cgatcacactgattgctaatgagcctgcaaacacatttgatgttatagaagctgcaaccaaagctaatcg

SEQ ID NO: 96 - DNA, 70 bp, Alstroemeria sp.
  source 1..70: mol_type = genomic DNA
gatggcctggaaggagatgctatggttctgcaattggagtcccagattctttaccgattaatggacatgg

SEQ ID NO: 97 - DNA, 70 bp, Alstroemeria sp.
  source 1..70: mol_type = genomic DNA
tctacaattcgagcaattactgatctaatgtcaaagggctgtctatgatcagttggcgcaatagatcgaa

SEQ ID NO: 98 - DNA, 70 bp, Alstroemeria sp.
  source 1..70: mol_type = genomic DNA
atatctcgaatcaattgctcgttggaaccgagatcgccctccgccaaagcttctctcaactcttcaatct

SEQ ID NO: 99 - DNA, 71 bp, Alstroemeria sp.
  source 1..71: mol_type = genomic DNA
gtgatggtggctgtatttagaattctaaaagactcgctttcagaaaaaaccttccaccttatagagttgct

SEQ ID NO: 100 - DNA, 70 bp, Alstroemeria sp.
  source 1..70: mol_type = genomic DNA
ccaatttccaggccatgaagctcatcatttgcgaagtgatctgaaacaccagatatcttacaatgcaagg

SEQ ID NO: 101 - DNA, 70 bp, Alstroemeria sp.
  source 1..70: mol_type = genomic DNA
actgcatcaagaagtttcttccattctcgtgcagcgtcaagacttgcatacacctcaaggtccttcagaa

SEQ ID NO: 102 - DNA, 70 bp, Alstroemeria sp.
  source 1..70: mol_type = genomic DNA
cacaatgctagtaacaaagttggagcatgcttcagctatgactgaatccacgcaaggaaggggtccatat

SEQ ID NO: 103 - DNA, 70 bp, Alstroemeria sp.
  source 1..70: mol_type = genomic DNA
gagcatctggtgtctgccgtagcaaatgtagagttgtctattatatgtagcttaccttcatcgatcacac

SEQ ID NO: 104 - DNA, 70 bp, Alstroemeria sp.
  source 1..70: mol_type = genomic DNA
atgccagcgcgacggatgagggcgaggtttagctcggcctcagttaggagcatgtcagcactaggattgc

SEQ ID NO: 105 - DNA, 70 bp, Alstroemeria sp.
  source 1..70: mol_type = genomic DNA
cgccaaccacaaaataatatccgttctggagactttggaccaataaaatccttgggcagatatcctccct

SEQ ID NO: 106 - DNA, 71 bp, Alstroemeria sp.
  source 1..71: mol_type = genomic DNA
tctcggataccatgatcaaatacctcttgatagtgtgctttccatgcagtatcatgggtggcgtaaaaaga

SEQ ID NO: 107 - DNA, 70 bp, Alstroemeria sp.
  source 1..70: mol_type = genomic DNA
acctcaatggagctacaacctggttctttctgaatcccttgctccttcatcattgcccgcatcctgacag

SEQ ID NO: 108 - DNA, 71 bp, Alstroemeria sp.
  source 1..71: mol_type = genomic DNA
accagtggtgtagctggaaatcctagaatggcacccgatgcagctgaaatacagcttaagagcccaaactg

SEQ ID NO: 109 - DNA, 70 bp, Alstroemeria sp.
  source 1..70: mol_type = genomic DNA
cagcctagtgaaacatagaagagcgtggtgaccccgatgccatacagggttgctctttgcattgttttgt

SEQ ID NO: 110 - DNA, 70 bp, Alstroemeria sp.
  source 1..70: mol_type = genomic DNA
gctcagcaatatggaggctctgctgcacataatactgttttcccagagaggcaagggaaatggaaggcta

SEQ ID NO: 111 - DNA, 70 bp, Alstroemeria sp.
  source 1..70: mol_type = genomic DNA
actggatcgaaggtcagtaaatgatccggcccgcggctcaggcagtagaccaatccatccagatatgccg

SEQ ID NO: 112 - DNA, 71 bp, Alstroemeria sp.
  source 1..71: mol_type = genomic DNA
gtgtcggaggagactggtaggaagaagttctctctgtgggcgtacgaggaatatgaaagtggaaacattgc

SEQ ID NO: 113 - DNA, 70 bp, Alstroemeria sp.
  source 1..70: mol_type = genomic DNA
cagtccggtaacacaattacaagttcttttaatacggagaaagctccaaccttggttttgatggactttt

SEQ ID NO: 114 - DNA, 70 bp, Alstroemeria sp.
  source 1..70: mol_type = genomic DNA
gcaagagatgcaatacaagaagctgacttctttctgacactggcttgattgacgaccaactgggatataa

SEQ ID NO: 115 - DNA, 70 bp, Alstroemeria sp.
  source 1..70: mol_type = genomic DNA
atcttatcttcacttgctactcctgaacaatctatggctataatggatctcattgaagcacgttgggaag

SEQ ID NO: 116 - DNA, 71 bp, Alstroemeria sp.
  source 1..71: mol_type = genomic DNA
catttcacaaggaactcaagatcccctttgttcaatgcagaattgtgaatatccatgatttcgcatggatg

SEQ ID NO: 117 - DNA, 70 bp, Alstroemeria sp.
  source 1..70: mol_type = genomic DNA
cacacatgtcaccagactccagttacaattgttcagtcagctttatctggtgaaatccagatgcttcttt

SEQ ID NO: 118 - DNA, 70 bp, Alstroemeria sp.
  source 1..70: mol_type = genomic DNA
ttgcactctcgtggtttcgtctgcggatgcttgacgccagcttgctttcgtcttgatgagtatgggcatt

SEQ ID NO: 119 - DNA, 70 bp, Alstroemeria sp.
  source 1..70: mol_type = genomic DNA
taagaacaattccgatgatcagaagagagttgcacggaatgactccgatggaacagacagtaacaaagag

SEQ ID NO: 120 - DNA, 70 bp, Alstroemeria sp.
  source 1..70: mol_type = genomic DNA
ccgaactacagtctggagctatggagcggacaactttggactctttggttgtgcagtacttgaagcacca

SEQ ID NO: 121 - DNA, 71 bp, Alstroemeria sp.
  source 1..71: mol_type = genomic DNA
cagggtaaaatatctgaagaattaatagatcgccttatggggactcttggtcatttggtgcagcgcaatac

SEQ ID NO: 122 - DNA, 70 bp, Alstroemeria sp.
  source 1..70: mol_type = genomic DNA
cgaaaaccctacccttatctcctcacaccgcaagatgctgcctttccatccaaaacctgtgatttctgga

SEQ ID NO: 123 - DNA, 70 bp, Alstroemeria sp.
  source 1..70: mol_type = genomic DNA
tcaacaaattcgggcttgatgtcatacaggaagcgtgatatcctatgccaaactcctttcggtgctagag

SEQ ID NO: 124 - DNA, 70 bp, Alstroemeria sp.
  source 1..70: mol_type = genomic DNA
ttccaaagtggagtccttcgtactaactgtatcgattgcttggatcgtacgaacgttgcacagtatgcat

SEQ ID NO: 125 - DNA, 70 bp, Alstroemeria sp.
  source 1..70: mol_type = genomic DNA
gcactaatttatgaccaagacgaaaacagtgaggtttacaagcattattctgaactatgtcagggacctg

SEQ ID NO: 126 - DNA, 71 bp, Alstroemeria sp.
  source 1..71: mol_type = genomic DNA
agccatctcaaaaagctgaacgcacactatttgttcatcgaatccctcaaaacgtcaataggccacgggcc

SEQ ID NO: 127 - DNA, 71 bp, Alstroemeria sp.
  source 1..71: mol_type = genomic DNA
atttcatgtgtgaaaattcatctccatccgaatatgtagcttcaaatggacttaacaacagacctgatgag

SEQ ID NO: 128 - DNA, 70 bp, Alstroemeria sp.
  source 1..70: mol_type = genomic DNA
aatgttggggcatgtggtgaagctgaaaagttgattgaaaatagcaaccctgtggagattcatttctcac

SEQ ID NO: 129 - DNA, 70 bp, Alstroemeria sp.
  source 1..70: mol_type = genomic DNA
ggagtagaatatcaggctgaacgtattttgcttgatctacagacgaatgacaaatattgtgatgaggact

SEQ ID NO: 130 - DNA, 71 bp, Alstroemeria sp.
  source 1..71: mol_type = genomic DNA
ataaatcaaaagagcgacaatatccattcttatcatgagattattaaggttgcaagacatttttaaaatct

SEQ ID NO: 131 - DNA, 70 bp, Alstroemeria sp.
  source 1..70: mol_type = genomic DNA
gtgttgcacccgagacgttatctgaagatggcattgctagagtttctaagacagagagcaaaagcgacac

SEQ ID NO: 132 - DNA, 70 bp, Alstroemeria sp.
  source 1..70: mol_type = genomic DNA
catcaacagaaacactgccatgctcaatggtgtagcattgtggttcattgaatctccagaaagaaatatt

SEQ ID NO: 133 - DNA, 70 bp, Alstroemeria sp.
  source 1..70: mol_type = genomic DNA
ttttattttcttaaaatgccgttctctcctgtaattgttttggtttctcgaaccgagatcacgatctcca

SEQ ID NO: 134 - DNA, 70 bp, Alstroemeria sp.
  source 1..70: mol_type = genomic DNA
tgacaaccgctgaacatgaacttcctatgagatttctgctttgaacctttattgggcacttctaaccctc

SEQ ID NO: 135 - DNA, 70 bp, Alstroemeria sp.; identical to SEQ ID NO: 131
  source 1..70: mol_type = genomic DNA

SEQ ID NO: 136 - DNA, 70 bp, Alstroemeria sp.
  source 1..70: mol_type = genomic DNA
gcgttggggatattctttagagcatgaaagcaatgtcagttaggtttgcttaggagtttaatgatctgtt

SEQ ID NO: 137 - DNA, 70 bp, Alstroemeria sp.
  source 1..70: mol_type = genomic DNA
aggcgtaaaggggtcaccggtaagaaagtagcaaatactacagctccaaagtccgcagaatgtagtaata

SEQ ID NO: 138 - DNA, 70 bp, Alstroemeria sp.
  source 1..70: mol_type = genomic DNA
tccaatccttcccctctttccacatcatggcatgattgtatcggtttgtaatgccaggctgcagaacttg

SEQ ID NO: 139 - DNA, 70 bp, Alstroemeria sp.
  source 1..70: mol_type = genomic DNA
tatacgagtagccatgttgataacccactatgccgtgaaagcttagatatggaactatcaactgctgcag

SEQ ID NO: 140 - DNA, 70 bp, Alstroemeria sp.
  source 1..70: mol_type = genomic DNA
tggtatacaagtatgtcagcgtgtgtccactgaaagcagaaagaaactgaaagcgatcagatattataat

SEQ ID NO: 141 - DNA, 70 bp, Alstroemeria sp.
  source 1..70: mol_type = genomic DNA
atacctttatataccttcccatatcctccttgtcccacttgagcagagttgctgaaattatttgtagcta

SEQ ID NO: 142 - DNA, 70 bp, Alstroemeria sp.
  source 1..70: mol_type = genomic DNA
cttgatccagaaaggtcgcttgtactgggaccttgtcctactagagtttgttgagaagaagctgttttcc

SEQ ID NO: 143 - DNA, 70 bp, Alstroemeria sp.
  source 1..70: mol_type = genomic DNA
gaatattgtgtacctctcgttcaaagggaagtacaggttataaagcaaaataacatcatgatcaggcctg

SEQ ID NO: 144 - DNA, 70 bp, Alstroemeria sp.
  source 1..70: mol_type = genomic DNA
tctgcttcggcgattcatcgtccaaatcatcggagggaggtgagctcatcaacttcccagcaacagtgtg

SEQ ID NO: 145 - DNA, 70 bp, Alstroemeria sp.
  source 1..70: mol_type = genomic DNA
atactatccccaggctctaatgttatatgcttagcgtccaatgatgagagagctttggtccatggccttc

SEQ ID NO: 146 - DNA, 70 bp, Alstroemeria sp.
  source 1..70: mol_type = genomic DNA
atttgatgaggatcttgtttcatgcgttttgaagcggttattactttcaaatggccaagggctgatgcaa

SEQ ID NO: 147 - DNA, 70 bp, Alstroemeria sp.
  source 1..70: mol_type = genomic DNA
ccttctaatctgttcctcaatattcttcctctatcgcagacaaaataaaataaacaactcagagcagatg

SEQ ID NO: 148 - DNA, 70 bp, Alstroemeria sp.
  source 1..70: mol_type = genomic DNA
ctgcgtccagtttggttgaagtaatctttgcagtcgctcataatcgcatccaaaggataggcctttgcac

SEQ ID NO: 149 - DNA, 70 bp, Alstroemeria sp.
  source 1..70: mol_type = genomic DNA
aaggagatcaaagaaaatatatttactatcatcgtgtacatcatcagttccaatattagatgcatccagt

SEQ ID NO: 150 - DNA, 70 bp, Alstroemeria sp.
  source 1..70: mol_type = genomic DNA
tgatttgatggctgcgttgacaaatttaaacctgatccattgagcaatttcctcggatgggaaagcaagg

SEQ ID NO: 151 - DNA, 70 bp, Alstroemeria sp.
  source 1..70: mol_type = genomic DNA
atgtcatcagattcatatctccagttctgctcatccttccgataactaggcatggatgaacttgatctcc

SEQ ID NO: 152 - DNA, 70 bp, Alstroemeria sp.
  source 1..70: mol_type = genomic DNA
aaccttatggccacacgcaatctcaacactttgagtatgccgttagtgagccaacctgttacatgtgaac

SEQ ID NO: 153 - DNA, 70 bp, Alstroemeria sp.
  source 1..70: mol_type = genomic DNA
gagagacctgattggctgttgttctcatggatcaataaagatgaagatgatgatctggcccttccaaata

SEQ ID NO: 154 - DNA, 70 bp, Alstroemeria sp.
  source 1..70: mol_type = genomic DNA
ttgtcaaggtagtcggccctgtaaatctttccagttgtagcttcattagcaatgttttcccagcgaattg

SEQ ID NO: 155 - DNA, 70 bp, Alstroemeria sp.
  source 1..70: mol_type = genomic DNA
tttggaaagctgctttaagttccacaagagcatcataatttatttcttctaggtaatgaaaatgtggaac

SEQ ID NO: 156 - DNA, 70 bp, Alstroemeria sp.
  source 1..70: mol_type = genomic DNA
gctgctgccctaggaagggtcctaatgctggaccttgtaattaggaatgaagatagacttccttgtcgcc

SEQ ID NO: 157 - DNA, 71 bp, Alstroemeria sp.
  source 1..71: mol_type = genomic DNA
tgattagtttattggatgaggttatgggatagtaacatttctattttgtgtagctgatttgtggtcctggt

SEQ ID NO: 158 - DNA, 70 bp, Alstroemeria sp.
  source 1..70: mol_type = genomic DNA
aagtggtcgctagagttcgtgagggtcttggtcatgccaccaacaaccaggctgaatatgagggcttgaa

SEQ ID NO: 159 - DNA, 70 bp, Alstroemeria sp.
  source 1..70: mol_type = genomic DNA
cacaaggcctctcttcctatatagcgtttgatgagcagctcattccatttaagctcttccttccatacaa

SEQ ID NO: 160 - DNA, 70 bp, Alstroemeria sp.
  source 1..70: mol_type = genomic DNA
gcaaagctactatatcaagagcttttcagcgtaggcatatgtctcaagtttgaaacagaggttcgttaaa

SEQ ID NO: 161 - DNA, 70 bp, Alstroemeria sp.
  source 1..70: mol_type = genomic DNA
gatttaagtgtatcaagtagccatgacacaagatctggaaagtgttcctcgcccattcctttgataagag

SEQ ID NO: 162 - DNA, 70 bp, Alstroemeria sp.
  source 1..70: mol_type = genomic DNA
agctgctacaagtaataatggcaaccattggtttcgtattcgtgtacctctgcatcactaaaggcgaaga

SEQ ID NO: 163 - DNA, 70 bp, Alstroemeria sp.
  source 1..70: mol_type = genomic DNA
ttctcggagtagacaacattcgttgcagggtcgaccgtggacttaatggcgtcgagaattgtggttccag

SEQ ID NO: 164 - DNA, 70 bp, Alstroemeria sp.
  source 1..70: mol_type = genomic DNA
cgtcggacagcttgtaactgtacagtaatttatcttttctctacgtttctcttgagatcatggattgaac

SEQ ID NO: 165 - DNA, 71 bp, Alstroemeria sp.
  source 1..71: mol_type = genomic DNA
ttcctttgccgaaagctgtgccctccgattaaagggatggatgtcagccctcttattgcattttgggtcat

SEQ ID NO: 166 - DNA, 70 bp, Alstroemeria sp.
  source 1..70: mol_type = genomic DNA
tctcaggcggtccgcgtcagcccgttccaaggaattgagcccaagtgcagtagttcacgcttctacgttg

SEQ ID NO: 167 - DNA, 70 bp, Alstroemeria sp.; identical to SEQ ID NO: 133
  source 1..70: mol_type = genomic DNA

SEQ ID NO: 168 - DNA, 70 bp, Alstroemeria sp.; identical to SEQ ID NO: 137
  source 1..70: mol_type = genomic DNA

SEQ ID NO: 169 - DNA, 71 bp, Alstroemeria sp.
  source 1..71: mol_type = genomic DNA
atggtaaaattttcagagaacttttttacagctgcgtctatgtttttcttctgattatatccaacggtaaa

SEQ ID NO: 170 - DNA, 70 bp, Alstroemeria sp.
  source 1..70: mol_type = genomic DNA
cacaacccattgatcaacacacaaaacgtgtcaacgctcggaacaaaatgtttcaacttcatgtacttcc

SEQ ID NO: 171 - DNA, 70 bp, Alstroemeria sp.
  source 1..70: mol_type = genomic DNA
cacagattgtcagacaggttatatcaaagttccccgggataaggttaatatacaaacgacactaatgatg

SEQ ID NO: 172 - DNA, 70 bp, Alstroemeria sp.
  source 1..70: mol_type = genomic DNA
ggaccagagggaaaggaaagacaactgaaagtaccgcagaaaatgaaagggctgtagtctctccaagttc

SEQ ID NO: 173 - DNA, 70 bp, Alstroemeria sp.
  source 1..70: mol_type = genomic DNA
tcctcttcctcttggccttcattgtcagtatcctcgtccatgttgtcagtgaaattcgggtcatagctta

SEQ ID NO: 174 - DNA, 70 bp, Alstroemeria sp.
  source 1..70: mol_type = genomic DNA
cttgccaacaagactagagacttctctggaacctgtacaaagacacgtcaatatgaacttcaagctatac

SEQ ID NO: 175 - DNA, 71 bp, Alstroemeria sp.
  source 1..71: mol_type = genomic DNA
ttgttatctccagctgccaagcaaagtacagcaacgcattgtgctattgaacaaagagcctgcttagccaa

SEQ ID NO: 176 - DNA, 71 bp, Alstroemeria sp.
  source 1..71: mol_type = genomic DNA
ctctcggaggcaggaggattgcctgtggttgtccttgcatctcgcttgcagacggggcaaaatgttctcca

SEQ ID NO: 177 - DNA, 70 bp, Alstroemeria sp.
  source 1..70: mol_type = genomic DNA
ctgatccttcagcagaagtcgtgggtatgtatgaggtggtcggggagggtgctgatctcactgatggatg

SEQ ID NO: 178 - DNA, 71 bp, Alstroemeria sp.
  source 1..71: mol_type = genomic DNA
gatgctcttcacaccatgcatgaagggaaattccttcatctccctgttgtagacagagatgggaacattgt

SEQ ID NO: 179 - DNA, 70 bp, Alstroemeria sp.; identical to SEQ ID NO: 103
  source 1..70: mol_type = genomic DNA

SEQ ID NO: 180 - DNA, 70 bp, Alstroemeria sp.
  source 1..70: mol_type = genomic DNA
atgctctacgctcagattgattcagtttacgcggcgatgaaagcaatggggcatacagacattggtgtca